(19) United States
(12) Patent Application Publication
(10) Pub. No.: US 2021/0246219 A1
(43) Pub. Date: Aug. 12, 2021

(54) COMBINATION THERAPIES COMPRISING CD137/HER2 BISPECIFIC AGENTS AND PD-1 AXIS INHIBITORS AND USES THEREOF

(71) Applicant: Pieris Pharmaceuticals GmbH, Hallbergmoos (DE)

(72) Inventors: Ingmar Bruns, Boston, MA (US); Louis Matis, Boston, MA (US); Shane Olwill, Freising (DE); Thomas Jaquin, Freising (DE)

(21) Appl. No.: 17/271,149

(22) PCT Filed: Aug. 27, 2019
(86) PCT No.: PCT/EP2019/072754

§ 371 Date: Feb. 24, 2021

(30) Foreign Application Priority Data
Aug. 27, 2018 (EP) 18191041.5

(51) Int. Cl. C07K 16/28; C07K 16/32; A61P 35/00

(52) U.S. Cl. CPC C07K 16/2878; C07K 16/32; C07K 16/2827; A61P 35/00; C07K 16/2818; A61K 2039/507; C07K 2317/31; C07K 2317/74; C07K 2317/76; C07K 2319/00; C07K 2317/41; C07K 2317/565

(57) ABSTRACT

The disclosure provides compositions and methods for treating previously treated specific HER2-positive advanced or metastatic solid tumors. The disclosure provides compositions and methods for enhancing immune response in an individual having HER2-positive advanced or metastatic solid tumors. The method comprises administering a PD-laxis inhibitor and a bispecific agent that targets CD137 and HER2.

### I. BACKGROUND

CD137, also known as 4-1BB, is a costimulatory immune receptor and a member of the tumor necrosis factor receptor (TNFR) super-family. CD137 plays an important role in the regulation of immune responses and thus is a target for cancer immunotherapy. CD137 ligand (CD137L) is the only known natural ligand of CD137 and is constitutively expressed on several types of antigen presenting cells (APCs), such as activated B cells, monocytes, and splenic dendritic cells, and it can be induced on T lymphocytes.

The potential of CD137 costimulation in cancer therapy has been demonstrated in many preclinical studies—the administration of agonist anti-CD137 antibodies have been shown to achieve tumor regression (Vinay and Kwon, Mol Cancer Ther, 2012, Bartkowiak and Curran, Front Oncol, 2015) and the resulting CD137 signaling to break and reverse the anergy in cytotoxic T lymphocytes (Williams et al., J Exp Med, 2017, Wilcox et al., Blood, 2004). Clinical trials of two agonist antibodies, urelumab and utomilumab, are ongoing, but both present significant challenges as urelumab has substantial toxicity at doses above 1 mg/kg, while utomilumab is less potent and has potential efficacy challenges (Tolcher et al., Clin Cancer Res, 2017, Segal et al., Clin Cancer Res, 2018, Sznol et al., Journal of Clinical Oncology, 2008). In addition, since the expression of CD137 is not limited to tumor infiltrating lymphocytes, urelumab or utomilumab monotherapy may not be capable of restricting CD137 agonism to a tumor microenvironment and thus may lead to CD137 clustering and activation in a non-localized manner (Makkouk et al., Eur J Cancer, 2016, Alizadeh et al., Blood, 2011). Therefore, there remains a need for anti-CD137 therapies that are both safe and effective. In addition, targeting CD137 in combination with other checkpoint immunotherapies or tumor-targeted therapies are being evaluated, but combining such therapies can augment the risk of undesired side effects, including increased systemic immune activation. Accordingly, there remains a need for combination immunotherapies that target CD137 and that are both safe and effective.

Programmed cell death protein 1, or PD-1 (also known as cluster of differentiation 279 or CD279) is a member of the cluster of differentiation 28 (CD28) gene family and is expressed on activated T, B, and myeloid lineage cells (Sharpe et al., Nat Immunol, 2007, Greenwald et al., Annu Rev Immunol, 2005). PD-1 interacts with two ligands, programmed cell death 1 ligand 1 (PD-L1) and programmed cell death 1 ligand 2 (PD-L2). Interaction of these ligands with PD-1 plays an important role in downregulating the immune system by limiting overly-active T cells locally, which in turn prevents autoimmunity and maintains peripheral tolerance during infection or inflammation in normal tissues (Sharpe et al., Nat Immunol, 2007, Greenwald et al., Annu Rev Immunol, 2005).

In many cancers, PD-1 is expressed by tumor-infiltrating lymphocytes (TILs) and is associated with host anti-tumor immunity (Galon et al., Science, 2006). Multiple lines of evidence have indicated that TILs are subject to PD-1 inhibitory regulation and that observed anti-tumor immunity is modulated by PD-1/PD-L1 signaling. First, PD-L1 expression is confirmed in several human and mouse tumor lines (Dong et al., Nat Med, 2002). Second, expression of PD-L1 by tumor cells has been directly associated with their resistance to lysis by anti-tumor T cells in vitro (Blank et al., Cancer Res, 2004, Dong et al., Nat Med, 2002). Third, PD-1 knockout mice are resistant to tumor challenge (lwai et al., Int Immunol, 2005) and T cells from PD-1 knockout mice are highly effective in tumor rejection when adoptively transferred to tumor-bearing mice (Blank et al., Cancer Res, 2004). Fourth, blocking PD-1 inhibitory signals by a PD-1 monoclonal antibody can potentiate host anti-tumor immunity in mice (Hirano et al., Cancer Res, 2005, lwai et al., Int Immunol, 2005). Fifth, high degrees of PD-L1 expression in tumors (detected by immunohistochemical staining) are associated with poor prognosis for many human cancer types (Hamanishi et al., Proc Natl Acad Sci USA, 2007). Accordingly, monoclonal antibodies targeting PD-1 and PD-L1 have demonstrated clinical benefits in various cancer types, with a few including nivolumab, pembrolizumab, and atezolizumab been approved by the U.S. Food and Drug Administration (FDA) (Topalian et al., Cancer Cell, 2015). However, only a minority of cancer patients respond to anti-PD-1/PD-L1 therapy and both primary and acquired resistance becomes a major obstacle of anti-PD-1/PD-L1 therapy, limiting the long-lasting effects and wide applications (Bu et al., Trends Mol Med, 2016, Bai et al., Oncotarget, 2017).

To increase the fraction of patients that respond to immunotherapy and overcome treatment resistance, there remains a need to maximize the clinical benefit of immune checkpoint treatment by the combination with, e.g., other immunotherapy or targeted therapy. However, while numerous preclinical studies are performed and demonstrated the potential of immunotherapy combinations, such combinations may result in substantive incremental toxicity, for example, as seen in the combination of anti-PD-1/PD-L1 therapy and ani-CTLA-4 therapy, and do not necessarily yield sufficient clinical responses to overcome the toxicities encountered (Shoushtari et al., JAMA Oncol, 2018, Haanen et al., Ann Oncol, 2017). Besides, for a specific immunotherapy combination, there is also the need to optimize the timing and sequence of the drugs' administration to achieve efficacy and safety and to identify better biological markers (biomarkers) given the diversity of patient response for predicting how individuals will react to the specific combination.

HER2, or HER2/neu, is a member of the human epidermal growth factor receptor family. Amplification or overexpression of this oncogene has been shown to play an important role in the development and progression of a variety of tumors, including certain aggressive types of breast cancer. HER2 has been shown to be highly differentially expressed on certain tumor cells, with much higher cell-surface density on those cells compared to healthy tissue. While anti-HER2 targeted therapy is effective in a portion of the patient population with early stage or metastatic HER2(+) breast cancer, the response rates among patients with either refractory or advanced cancer are suboptimal. For example, the objective response rate in a clinical trial using an anti-HER2 therapy with chemotherapy is only 50% (Slamon et al., N Engl J Med, 2001). Therefore, there remains a need for better targeted therapy for patient with HER2 positive cancer.

The present disclosure provides, among other things, novel therapeutic combinations comprising a CD137/HER2 bispecific agent and a PD-1 axis inhibitor and methods for treating HER2-positive advanced or metastatic solid tumors using such combinations.

### II. DEFINITIONS

The following list defines terms, phrases, and abbreviations used throughout the instant specification. All terms listed and defined herein are intended to encompass all grammatical forms.

As used herein, unless otherwise specified, “CD137” means human CD137 (huCD137). Human CD137 means a full-length protein defined by UniProt Q07011, a fragment thereof, or a variant thereof. CD137 is also known as 4-1 BB, tumor necrosis factor receptor superfamily member 9 (TNFRSF9), and induced by lymphocyte activation (ILA). In some particular embodiments, CD137 of non-human species, e.g., cynomolgus CD137 and mouse CD137, is used.

As used herein, unless otherwise specified, “HER2” means human HER2 (huHER2). Human Her 2 means a full-length protein defined by UniProt P04626, a fragment thereof, or a variant thereof. HER2 is also known as human epidermal growth factor receptor 2, HER2/neu, receptor tyrosine-protein kinase erbB-2, cluster of differentiation 340 (CD340), proto-oncogene Neu, ERBB2 (human), Erbb2 (rodent), c-neu, or p185. Human HER2 is encoded by the ERBB2 gene. In some particular embodiments, HER2 of non-human species, e.g., cynomolgus HER2 and mouse HER2, is used.

As used herein, unless otherwise specified, “programmed cell death protein 1” or “PD-1” means human PD-1 (huPD-1). Human PD-1 means a full-length protein defined by UniProt Q15116, a fragment thereof, or a variant thereof. Human PD-1 is encoded by the PDCD1 gene. PD-1 is also known as cluster of differentiation 279 or CD279. In some particular embodiments, PD-1 of non-human species, e.g., cynomolgus PD-1 and mouse PD-1, is used.

As used herein, unless otherwise specified, “programmed cell death 1 ligand 1” or “PD-L1” means human PD-L1 (huPD-L1). Human PD-L1 means a full-length protein defined by UniProt Q9NZQ7, a fragment thereof, or a variant thereof. Human PD-L1 is encoded by the CD274 gene. PD-L1 is also known as cluster of differentiation 274 (CD274) or B7 homolog 1 (B7-H1). In some particular embodiments, PD-L1 of non-human species, e.g., cynomolgus PD-L1 and mouse PD-L1, is used.

As used herein, “PD-1 axis inhibitor” or “PD-1 axis antagonist” refers to a molecule that decreases, blocks, inhibits, or interferes with PD-1-mediated signaling so that to alleviate T cell dysfunction and immune suppression resulting from the activation of the PD-1 axis and restore or enhance T cell functions. As used herein, a PD-1 axis inhibitor includes a PD-1 inhibitor, a PD-L1 inhibitor, and a PD-L2 inhibitor.

As used herein, “PD-1/PD-L1 axis inhibitor” or “PD-1/PD-L1 axis inhibitor” refers to a molecule that decreases, blocks, inhibits, or interferes with the interaction between PD-1 and PD-L1 and the resulting signaling pathways, so that to alleviate T cell dysfunction and immune suppression resulting from the activation of the PD-1 axis and restore or enhance T cell functions.

As used herein, “PD-1 inhibitor” or “PD-1 antagonist” refers to a molecule that decreases, blocks, inhibits, interferes with the interaction of PD-1 with one or more of its binding partners, such as PD-L1, PD-L2, and so as the resulting signaling pathways. In some embodiments, a PD-1 inhibitor may compete with the binding of one or more PD-1 binding partners, preferably PD-L1 and/or PD-L2. In some other embodiments, a PD-1 inhibitor may not compete with the binding of one or more PD-1 binding partners, preferably PD-L1 and/or PD-L2. In some embodiments, a PD-1 inhibitor may reduce the negative costimulatory signal mediated by or through cell surface proteins expressed on T lymphocytes, such as PD-1. PD-1 inhibitors may include, but not limited to, anti-PD-1 antibodies and antigen binding fragments thereof, immunoadhesins, fusion proteins, oligopeptides, and other molecules that decrease, block, inhibit, or interfere with the interaction of PD-1 with one or more of its binding partners, such as PD-L1 and PD-L2, and so as the resulting signaling pathways. In some embodiments, a PD-1 inhibitor may be or comprise an anti-PD-1 antibody, such as pembrolizumab, nivolumab, pidilizumab, MK-3475, MEDI-0680, PDR001, REGN2810, BGB-108.

As used herein, “PD-L1 inhibitor” or “PD-L1 antagonist” refers to a molecule that decreases, blocks, inhibits, interferes with the interaction of PD-L1 with one or more of its binding partners, such as PD-1 and B7.1, and so as the resulting signaling pathways. In some embodiments, a PD-L1 inhibitor may compete with the binding of one or more PD-L1 binding partners, such as PD-1 and B7.1. In some other embodiments, a PD-L1 inhibitor may not compete with the binding of one or more PD-L1 binding partners, such as PD-1 and/or B7.1. In some embodiments, a PD-L1 inhibitor may reduce the inhibitory signal mediated by the binding of PD-L1 to one or more of its binding partners. In some embodiments, a PD-L1 inhibitor may be or comprise an anti-PD-L1 antibody, such as atezolizumab, avelumab, durvalumab, and BMS-936559.

As used herein, “PD-L2 inhibitor” or “PD-L2 antagonist” refers to a molecule that decreases, blocks, inhibits, interferes with the interaction of PD-L2 with one or more of its binding partners, such as PD-1, and so as the resulting signaling pathways. In some embodiments, a PD-L2 inhibitor may compete with the binding of one or more PD-L2 binding partners, such as PD-1. In some other embodiments, a PD-L2 inhibitor may not compete with the binding of one or more PD-L2 binding partners, such as PD-1. In one embodiment, a PD-L2 inhibitor may reduce the inhibitory signal mediated by the binding of PD-L1 to one or more of its binding partners. In some embodiments, a PD-L2 inhibitor may be or comprise an anti-PD-L2 antibody.

The term “anti-”, when used to describe a molecule in association with a protein target of interest (e.g., CD137, PD-1, PD-L1, or HER2), means the molecule is capable of binding the protein target and/or modulating one or more biological functions of the protein target. For example, an “anti-CD137” molecule as described herein, is capable of binding CD137 and/or modulating one or more biological functions of CD137. “Biological function” of a protein target refers to the ability of the protein target to carry out its biological mission(s), e.g., binding to its binding partner(s) and mediating signaling pathway(s).

As used herein, “dysfunction” or “dysfunctional” when used in connection with T cells, refers to dysfunctional states of T cells as a consequence of altered activation and differentiation processes. Terms including exhaustion, tolerance, and anergy may be used to describe the dysfunctional states. When used in the connection with the immune system, “dysfunction” refers to states of immune system characterized by abnormality in the components of the immune system and/or the overactive or underactive immune responses. Dysfunctional states of the immune system may include refractory or unresponsive to antigen recognition and impaired capacity to translate antigen recognition into downstream T-cell effector functions, such as proliferation, cytokine (e.g., IL-2) production, and target cell killing.

As used herein, “anergy” or “anergic” refers to a state where the immune system is unable to mount a normal immune response against a antigen. T cell anergy may be induced by incomplete or insufficient signals delivered through T cell receptors (TCRs). T cell anergy may also be induced when T cell receives TCR signals in the absence of costimulation, resulting in the cell becoming refractory to subsequent activation by the antigen even in the context of costimulation. In some embodiments, anergic T cells do not undergo clonal expansion and/or acquire effector functions. The anergic state as described herein may, in some embodiments, be overridden by the presence of IL-2.

As used herein, “exhaustion” or “T cell exhaustion” refers to a state of T cell dysfunction that arises from sustained TCR signaling. Exhaustion may result from both extrinsic negative regulatory pathways (e.g., immunoregulatory cytokines), as well as cell intrinsic negative regulatory. It may be characterized by poor effector function, sustained expression of inhibitory receptors, and/or a transcriptional state distinct from that of functional effector or memory T cells. In some embodiments, exhaustion is different from anergy in that it arises from continuous TCR stimulation, while anergy is introduced due to incomplete or deficient signaling. In some embodiments, exhaustion may occur during many chronic infections and cancers.

As used herein, “tolerance” includes “central tolerance” and “peripheral tolerance”. “Central tolerance” refers to a process of eliminating any developing T or B lymphocytes. In some embodiments, the process may ensure that the immune system does not attack self peptides. “Peripheral tolerance” refers to the second branch of immunological tolerance after central tolerance, which takes place in the immune periphery. In some embodiments, peripheral tolerance may ensure that self-reactive T and B cells which escaped central tolerance do not cause autoimmunity.

As used herein, “inhibitory signal” refers to signals transduced to T cells by co-inhibitory molecules, which lead to inhibition of T cell cytokine production and/or effector function. “Stimulatory signal” refers to signals transduced to T cells by costimulatory molecules following TCR engagement, rescuing the activated T cell from anergy and/or allowing full activation to occur.

As used herein, “restore T-cell function”, “enhance T-cell activity”, “activate T cells”, or “stimulate T-cell response” refers to induce, cause, or stimulate T cells to have a sustained or amplified biological functions, or renew or reactivate exhausted or inactive T cells. Exemplary signs of enhanced T-cell activity may include: increased secretion of INF-gamma from CD8⁺ T cells, increased proliferation, and/or increased antigen responsiveness (e.g., viral, pathogen, and tumor clearance) relative to such levels before the intervention. Methods of measuring such enhancement is known to the skilled in the art.

As used herein, “synergy” or “synergistic” refers to the interaction or cooperation of two or more substances, drugs, or other agents to produce a combined effect greater than the sum of their separate effects.

As used herein, “cancer” and “cancerous” refers to the physiological condition in mammals that is typically characterized by unregulated cell growth. A “tumor” may comprise one or more cancerous cells.

The term “metastatic” refers to a state of cancer where the cancer cells break away from where they first formed, travel through the blood or lymph system, and/or form new tumors (metastatic tumors) in other parts of the body. An “advanced” cancer is one that has spread outside the site or organ of origin, either by local invasion or metastasis.

As used herein, an “anti-tumor agent” may act on tumor, particularly malignant tumor (cancerous), and preferably has an anti-tumor effect or anti-tumor activity. The “anti-tumor effect” or “anti-tumor activity” refers to actions of an anti-tumor agent on tumor, particularly malignant tumor, including stimulation of tumor-specific immune responses, reduction in tumor size, suppression of the growth of tumor cells, suppression of the metastasis, complete remission, partial remission, stabilization of disease, extension of the term before recurrence, extension of survival time of patients, or improvement of quality of life of patients.

As used herein, “treat” or “treatment” refers to clinical intervention designed to alter the natural course of the individual or cell being treated during the course of a physiological condition or disorder or clinical pathology. A “treatment” refers to both therapeutic treatment and prophylactic or preventative measures, wherein the object is to prevent or slow down (lessen) an undesired physiological change or disorder, such as the growth, development or spread of a hyperproliferative condition, such as cancer. Desired effects of treatment include, but not limited to, decreasing the rate of disease progression, ameliorating or palliating the disease state, alleviating symptoms, stabilizing or not worsening the disease state, and remission of improved prognosis, whether detectable or undetectable. Desired effects of treatment also include prolonging survival as compared to expected survival if not receiving treatment. A subject in need of treatment include a subject already with the condition or disorder or prone to have the condition or disorder or a subject in which the condition or disorder is to be prevented.

As used herein, “combination”, “in combination with”, or “in conjunction with” relates to administration of one substance, drug, or other agent in addition to another substance, drug, or other agent. The administration of substance, drug, or other agent may be before, during, or after administration of the other.

An “effective amount” is an amount sufficient to effect beneficial or desired results. For example, an effective amount is one that would be sufficient to enhance or diminish the immune response to a desired level of a therapy. The results of a therapy (e.g., activation of a suppressed or deficient immune response, increased cytolytic activity of T cells, increased T cell effector function, or reduction in tumor growth) can be determined by suitable methods known in the art. An effective amount can be administered in one or more individual administrations or doses. An effective amount can be administered alone with one agent or in combination with one or more additional agents.

As used herein, “antibody” includes whole antibodies or any antigen binding fragment (i.e., “antigen-binding portion”) or single chain thereof. A whole antibody refers to a glycoprotein comprising at least two heavy chains (HCs) and two light chains (LCs) inter-connected by disulfide bonds. Each heavy chain is comprised of a heavy chain variable domain (V_{H} or HCVR) and a heavy chain constant region (C_{H}). The heavy chain constant region is comprised of three domains, C_{H1}, C_{H2} and C_{H3}. Each light chain is comprised of a light chain variable domain (V_{L} or LCVR) and a light chain constant region (C_{L}). The light chain constant region is comprised of one domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} is composed of three CDRs and four FRs, arranged in the following order from the amino-terminus to the carboxy-terminus: FR1, CDR1, FR2, CDR2, FR3, CDR3, FR4. The variable regions of the heavy and light chains contain a binding domain that interacts with an antigen (for example, PD-L1). The constant regions of the antibodies may optionally mediate the binding of the immunoglobulin to host tissues or factors, including various cells of the immune system (e.g., effector cells) and the first component (C1q) of the classical complement system.

As used herein, “antigen-binding domain” or “antigen-binding fragment” of an antibody refers to one or more fragments of an antibody that retain the ability to specifically bind to an antigen (e.g., PD-1 or PD-L1). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H1} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between C_{H1} and C_{H2} domains; (iv) a Fd fragment consisting of the V_{H} and C_{H1} domains; (v) a single-chain Fv fragment consisting of the V_{H} and V_{L} domains of a single arm of an antibody, (vi) a dAb fragment (Ward et al., Nature, 1989) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., Proc Natl Acad Sci USA, 1993); (ix) a “domain antibody fragment” containing only the V_{H} or V_{L}, where in some instances two or more V_{H} regions are covalently joined.

As used herein, “antigen binding fragment” of an antibody refers to one or more fragments of an antibody that retain the ability to specifically bind to an antigen (e.g., PD-L1). It has been shown that the antigen-binding function of an antibody can be performed by fragments of a full-length antibody. Examples of binding fragments encompassed within the term “antigen-binding fragment” of an antibody include (i) a Fab fragment consisting of the V_{H}, V_{L}, C_{L} and C_{H1} domains; (ii) a F(ab′)₂ fragment comprising two Fab fragments linked by a disulfide bridge at the hinge region; (iii) a Fab′ fragment consisting of the V_{H}, V_{L}, C_{L} and C_{H1} domains and the region between C_{H1} and C_{H2} domains; (iv) a Fd fragment consisting of the V_{H} and C_{H1} domains; (v) a single-chain Fv fragment consisting of the V_{H} and V_{L} domains of a single arm of an antibody, (vi) a dAb fragment (Ward et al., Nature, 1989) consisting of a V_{H} domain; and (vii) an isolated complementarity determining region (CDR) or a combination of two or more isolated CDRs which may optionally be joined by a synthetic linker; (viii) a “diabody” comprising the V_{H} and V_{L} connected in the same polypeptide chain using a short linker (see, e.g., patent documents EP 404,097; WO 93/11161; and Holliger et al., Proc Natl Acad Sci USA, 1993); (ix) a “domain antibody fragment” containing only the V_{H} or V_{L}, where in some instances two or more V_{H} regions are covalently joined.

Antibodies may be polyclonal or monoclonal; xenogeneic, allogeneic, or syngeneic; or modified forms thereof (e.g., humanized, chimeric, or multispecific). Antibodies may also be fully human.

As used herein, “framework” or “FR” refers to the variable domain residues other than the hypervariable region (CDR) residues.

“Fragment crystallizable region” or “Fc region” refers to the C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof numbering according to EU index of Kabat (Johnson and Wu, Nucleic Acids Res, 2000). The C-terminal lysine (residue 447 according to EU index of Kabat) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the invention include human IgG1, IgG2 (IgG2A, IgG2B), IgG3, and IgG4.

“Fc receptor” or “FcR” refers to a receptor that binds to the Fc region of an antibody.

As used herein, “isolated antibody” refers to an antibody that is substantially free of its natural environment. For instance, an isolated antibody is substantially free of cellular material and other proteins from the cell or tissue source from which it is derived. An “isolated antibody” further refers to an antibody that is substantially free of other antibodies having different antigenic specificities. In the present case, an isolated antibody that binds specifically PD-L1 is substantially free of antibodies that specifically bind antigens other than PD-L1. However, an isolated antibody that specifically binds PD-L1 may have cross-reactivity to other antigens, such as PD-L1 molecules from other species.

As used herein, “monoclonal antibody” refers to a preparation of antibody molecules of single molecular composition. A monoclonal antibody composition displays a single binding specificity and affinity for a particular epitope.

As used herein, “humanized antibody” refers to an antibody that consists of the CDR of antibodies derived from mammals other than human, and the FR region and the constant region of a human antibody or derived from a human antibody. In some embodiments a humanized antibody comprises a variable domain that has a variable region amino acid sequence which, analyzed as a whole, is closer to human than to other species as assessed using the Immunogenetics Information System (IMGT) DomainGapAlign tool, as described by Ehrenmann et al. (2010). In some embodiments, a humanized antibody may be useful as an effective component in a therapeutic agent due to the reduced antigenicity. The term “therapeutic agent” or “therapeutically active agent”, as used herein, refers to an agent which is therapeutically useful. A therapeutic agent may be any agent for the prevention, amelioration, or treatment of a diseases, a physiological condition, a symptom, or for the evaluation or diagnosis thereof.

As used herein, “human antibody” includes antibodies having variable regions in which both the framework and CDR regions are derived from human germline immunoglobulin sequences. Furthermore, if the antibody contains a constant region, the constant region is also derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “effector functions” as used herein with respect to antibodies refer to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the term “lipocalin” refers to a monomeric protein of approximately 18-20 kDa in weight, having a cylindrical β-pleated sheet supersecondary structural region comprising a plurality of β-strands (preferably eight β-strands designated A to H) connected pair-wise by a plurality of (preferably four) loops at one end to thereby comprise a ligand-binding pocket and define the entrance to the ligand-binding pocket. Preferably, the loops comprising the ligand-binding pocket used in the present invention are loops connecting the open ends of β-strands A and B, C and D, E and F, and G and H, and are designated loops AB, CD, EF, and GH. It is well-established that the diversity of the said loops in the otherwise rigid lipocalin scaffold gives rise to a variety of different binding modes among the lipocalin family members, each capable of accommodating targets of different sizes, shape, and chemical character (reviewed, e.g. in Skerra, Biochim Biophys Acta, 2000, Flower et al., Biochim Biophys Acta, 2000, Flower, Biochem J, 1996). It is understood that the lipocalin family of proteins has naturally evolved to bind a wide spectrum of ligands, sharing unusually low levels of overall sequence conservation (often with sequence identities of less than 20%) yet retaining a highly conserved overall folding pattern. The correspondence between positions in various lipocalins is also well-known to one of skill in the art (see, e.g., U.S. Pat. No. 7,250,297). Proteins fall in the definition of “lipocalin” as used herein include, but not limited to, human lipocalins including tear lipocalin (Tlc, Lcn1), Lipocalin-2 (Lcn2) or neutrophil gelatinase-associated lipocalin (NGAL), apolipoprotein D (ApoD), apolipoprotein M, α₁-acid glycoprotein 1, α₁-acid glycoprotein 2, α₁-microglobulin, complement component 8γ, retinol-binding protein (RBP), the epididymal retinoic acid-binding protein, glycodelin, odorant-binding protein Ila, odorant-binding protein IIb, lipocalin-15 (Lcn15), and prostaglandin D synthase.

As used herein, “Lipocalin-2” or “neutrophil gelatinase-associated lipocalin” refers to human Lipocalin-2 (hLcn2) or human neutrophil gelatinase-associated lipocalin (hNGAL) and further refers to the mature human Lipocalin-2 or mature human neutrophil gelatinase-associated lipocalin. The term “mature” when used to characterize a protein means a protein essentially free from the signal peptide. A “mature hNGAL” of the instant disclosure refers to the mature form of human neutrophil gelatinase-associated lipocalin, which is free from the signal peptide. Mature hNGAL is described by residues 21-198 of the sequence deposited with the SWISS-PROT Data Bank under Accession Number P80188, and the amino acid of which is indicated in SEQ ID NO: 1.

As used herein, a “native sequence” refers to a protein or a polypeptide having a sequence that occurs in nature or having a wild-type sequence, regardless of its mode of preparation. Such native sequence protein or polypeptide can be isolated from nature or can be produced by other means, such as by recombinant or synthetic methods.

The “native sequence lipocalin” refers to a lipocalin having the same amino acid sequence as the corresponding polypeptide derived from nature. Thus, a native sequence lipocalin can have the amino acid sequence of the respective naturally-occurring (wild-type) lipocalin from any organism, in particular, a mammal. The term “native sequence”, when used in the context of a lipocalin specifically encompasses naturally-occurring truncated or secreted forms of the lipocalin, naturally-occurring variant forms such as alternatively spliced forms and naturally-occurring allelic variants of the lipocalin. The terms “native sequence lipocalin” and “wild-type lipocalin” are used interchangeably herein.

As used herein, a “mutein,” a “mutated” entity (whether protein or nucleic acid), or “mutant” refers to the exchange, deletion, or insertion of one or more amino acids or nucleotides, compared to the naturally-occurring (wild-type) protein or nucleic acid. Said term also includes fragments of a mutein as described herein. The present disclosure explicitly encompasses lipocalin muteins, as described herein, having a cylindrical β-pleated sheet supersecondary structural region comprising eight β-strands connected pair-wise by four loops at one end to thereby comprise a ligand-binding pocket and define the entrance of the ligand-binding pocket, wherein at least one amino acid of each of at least three of said four loops has been mutated as compared to the native sequence lipocalin. Lipocalin muteins of the present invention thereof preferably have the function of binding CD137 as described herein.

As used herein, the term “fragment,” in connection with the lipocalin muteins of the disclosure, refers to proteins or polypeptides derived from full-length mature hNGAL or lipocalin muteins that are N-terminally and/or C-terminally truncated, i.e., lacking at least one of the N-terminal and/or C-terminal amino acids. Such fragments may include at least 10 or more, such as 20 or 30 or more consecutive amino acids of the primary sequence of mature hNGAL or the lipocalin mutein it is derived and are usually detectable in an immunoassay of mature hNGAL. Such a fragment may lack up to 2, up to 3, up to 4, up to 5, up to 10, up to 15, up to 20, up to 25, or up to 30 (including all numbers in between) of the N-terminal and/or C-terminal amino acids. It is understood that the fragment is preferably a functional fragment of mature hNGAL or the lipocalin mutein from which it is derived, which means that it preferably retains the binding specificity, preferably to CD137, of mature hNGAL or lipocalin mutein it is derived from. As an illustrative example, such a functional fragment may comprise at least amino acids at positions 13-157, 15-150, 18-141, 20-134, 25-134, or 28-134 corresponding to the linear polypeptide sequence of mature hNGAL.

A “fragment” with respect to CD137, HER2, PD-1, or PD-L1, refers to N-terminally and/or C-terminally truncated CD137, HER2, PD-1, or PD-L1 or protein domains of CD137, HER2, PD-1, or PD-L1. Fragments of CD137, HER2, PD-1, or PD-L1 as described herein retain the capability of the full-length CD137, HER2, PD-1, or PD-L1 to be recognized and/or bound by provided a lipocalin mutein, an antibody, a fusion protein, and/or a combination thereof.

As used herein, “bispecific” refers to a molecule is able to specifically bind to at least two distinct targets. Typically, a bispecific molecule comprises two target-binding sites, each of which is specific for a different target. In some embodiments, the bispecific molecule is capable of simultaneously binding two targets.

As used interchangeably herein, the terms “conjugate,” “conjugation,” “fuse,” “fusion,” or “linked” refer to the joining together of two or more subunits, through all forms of covalent or non-covalent linkage, by means including, but not limited to, genetic fusion, chemical conjugation, coupling through a linker or a cross-linking agent, and non-covalent association.

The term “fusion polypeptide” or “fusion protein” as used herein refers to a polypeptide or protein comprising two or more subunits. In some embodiments, a fusion protein as described herein comprises two or more subunits, at least one of these subunits being capable of specifically binding to CD137, and a further subunit capable of specifically binding to HER2. Within the fusion protein, these subunits may be linked by covalent or non-covalent linkage. Preferably, the fusion protein is a translational fusion between the two or more subunits. The translational fusion may be generated by genetically engineering the coding sequence for one subunit in a reading frame with the coding sequence of a further subunit. Both subunits may be interspersed by a nucleotide sequence encoding a linker. However, the subunits of a fusion protein of the present disclosure may also be linked through chemical conjugation. The subunits forming the fusion protein are typically linked to each other C-terminus of one subunit to the N-terminus of another subunit, or C-terminus of one subunit to C-terminus of another subunit, or N-terminus of one subunit to N-terminus of another subunit, or N-terminus of one subunit to C-terminus of another subunit. The subunits of the fusion protein can be linked in any order and may include more than one of any of the constituent subunits. If one or more of the subunits is part of a protein (complex) that consists of more than one polypeptide chain, the term “fusion protein” may also refer to the protein comprising the fused sequences and all other polypeptide chain(s) of the protein (complex). As an illustrative example, where a full-length immunoglobulin is fused to a lipocalin mutein via a heavy or light chain of the immunoglobulin, the term “fusion protein” may refer to the single polypeptide chain comprising the lipocalin mutein and the heavy or light chain of the immunoglobulin. The term “fusion protein” may also refer to the entire immunoglobulin (both light and heavy chains) and the lipocalin mutein fused to one or both of its heavy and/or light chains.

As used herein, the term “subunit” of a fusion protein disclosed herein refers to a single protein or a separate polypeptide chain, which may form a stable folded structure by itself and define a unique function of providing binding motif towards a target. In some embodiments, a preferred subunit of the disclosure is a lipocalin mutein. In some other embodiments, a preferred subunit of the disclosure is a full-length immunoglobulin or an antigen-binding domain thereof.

A “linker” that may be comprised by a fusion protein of the present disclosure joins together two or more subunits of a fusion protein as described herein. The linkage can be covalent or non-covalent. A preferred covalent linkage is via a peptide bond, such as a peptide bond between amino acids. A preferred linker is a peptide linker. Accordingly, in a preferred embodiment, said linker comprises one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. Other preferred linkers include chemical linkers.

A “sample” is defined as a biological sample taken from any subject. Biological samples include, but are not limited to, blood, serum, urine, feces, semen, or tissue, including tumor tissue.

A “subject” is a vertebrate, preferably a mammal, more preferably a human. The term “mammal” is used herein to refer to any animal classified as a mammal, including, without limitation, humans, domestic and farm animals, and zoo, sports, or pet animals, such as sheep, dogs, horses, cats, cows, rats, pigs, apes such as cynomolgus monkeys, to name only a few illustrative examples. Preferably, the “mammal” used herein is human.

As used herein the term “about” or “approximately” means within 20%, preferably within 15%, preferably within 10%, and more preferably within 5% of a given value or range. It also includes the concrete number, i.e. “about 20” includes the number of 20.

### III. DESCRIPTIONS OF FIGURES

FIG. 1: shows the ability of the CD137/HER2 bispecific agent shown in SEQ ID NOs: 81 and 80 in combination with the PD-1 antibody shown in SEQ ID NOs: 72 and 73 to costimulate T-cell activation. In the experiment, tumor cell line NCI-N87 was cultured on a plate overnight. The next day, human peripheral blood mononuclear cells (PBMCs) were added to the plate and incubated with the CD137/HER2 bispecific agent (SEQ ID NOs: 81 and 80) alone, the PD-1 antibody (SEQ ID NOs: 72 and 73) alone, or a combination of the two, at the molar ratio of 1:10 in the presence of 0.05 ng/mL staphylococcal enterotoxin B (SEB). Levels of secreted interleukin 2 (IL-2), as marker of T-cell activation, were determined by an electrochemiluminescence-based assay and normalized to the levels of corresponding IgG4 isotype control antibody (SEQ ID NOs: 15 and 16), as described in Example 1. Results from two tested donors are depicted in FIG. 1A and 1B, respectively. The combination of the CD137/HER2 bispecific agent (SEQ ID NOs: 81 and 80) and the PD-1 antibody (SEQ ID NOs: 72 and 73) was able to induce a dose-dependent IL-2 secretion, with an improved (lower) EC₅₀, as compared to the CD137/HER2 bispecific agent or the PD-1 antibody alone.

FIG. 2: shows the ability of the CD137/HER2 bispecific agent shown in SEQ ID NOs: 81 and 80 in combination with a PD-1 antibody shown in SEQ ID NOs: 72 and 73 (FIG. 2A) or in SEQ ID NOs: 74 and 75 (FIG. 2B) to costimulate T-cell activation. In the experiment, tumor cell line NCI-N87 was cultured on a plate overnight. The next day, human PBMCs were added to the plate and incubated with the CD137/HER2 bispecific agent (SEQ ID NOs: 81 and 80) at various concentrations in combination with the PD-1 antibody shown in SEQ ID NOs: 72 and 73 or SEQ ID NOs: 74 and 75 at the fixed concentration of 10 nM or 100 nM, in the presence of 1 ng/mL of SEB. The CD137/HER2 bispecific agent (SEQ ID NOs: 81 and 80) and the PD-1 antibody (SEQ ID NOs: 72 and 73 or SEQ ID NOs: 74 and 75) were also tested alone for comparison. Levels of secreted IL-2 were determined by an electrochemiluminescence-based assay as readout for T-cell activation and normalized to the levels of corresponding IgG4 isotype control antibody (SEQ ID NOs: 15 and 16), as described in Example 2. Representative results of the CD137/HER2 bispecific agent (SEQ ID NOs: 81 and 80) tested at 10 nM and 3.33 nM are depicted. The combination of the CD137/HER2 bispecific agent (SEQ ID NOs 81 and 80:) and the PD-1 antibody shown in SEQ ID NOs: 72 and 73 and the combination of the CD137/HER2 bispecific agent (SEQ ID NOs: 81 and 80) and the PD-1 antibody shown in SEQ ID NOs: 74 and 75 both induced higher IL-2 secretion and act synergistically or additively. When combined with SEQ ID NOs: 72 and 73, the induced IL-2 secretion levels were higher when 100 nM antibody was used than when 10 nM antibody was used. On the other hand, the concentration (at 100 nM or 10 nM) of SEQ ID NOs: 74 and 75 did not affect the IL-2 secretion when used in combination with the CD137/HER2 bispecific agent.

FIG. 3: shows the results of representative experiments in which the ability of the combination of a CD137/HER2 bispecific agent shown in SEQ ID NOs: 81 and 80 and a PD-L1 antibody (SEQ ID NOs: 76 and 77) to induce T-cell activation was investigated. In the experiment, tumor cell line NCI-N87 (HER2 high, PD-L1 low), JIMT-1 (HER2 moderate, PD-L1 moderate) or MDA-MB-231 (HER2 low, PD-L1 moderate) was cultured on a plate overnight. The next day, human PBMCs were added to the plate and incubated with the CD137/HER2 bispecific agent (SEQ ID NOs: 81 and 80) at various concentrations, alone or in combination with the PD-L1 antibody (SEQ ID NOs: 76 and 77) or IgG4 isotype control antibody (SEQ ID NOs: 15 and 16) at 100 nM, in the presence of 0.05 ng/mL SEB. Levels of secreted IL-2 were determined as described in Example 3. Exemplary data of the CD137/HER2 bispecific agent (SEQ ID NOs: 81 and 80) tested at the concentration of 2.5 nM and 0.16 nM are shown in FIG. 3. The functional activity of the CD137/HER2 bispecific agent in combination with the PD-L1 antibody, measured by the ability to activate T cells or increase IL-2 secretion, is synergistic/additive in the presence of tumor cells expressing high or moderate level of HER2 (NCI-N87 and JIMT-1).

FIG. 4: describes the overall study design of a Phase 1b study to determine the maximum tolerated dose (MTD) and recommended Phase 2 dose (RP2D) and assess the safety, efficacy, and PK of a CD137/HER2 bispecific agent shown in SEQ ID NOs: 81 and 80 administered in combination with a PD-L1 antibody shown in SEQ ID NOs: 76 and 77 in specific advanced or metastatic HER-2 positive solid tumors (e.g., bladder, breast and gastrointestinal). The study includes a dose escalation period followed by an expansion period.

FIG. 5. provides an overview over the design of representative CD137/HER2 bispecific fusion proteins as described herein. Representative CD137/HER2 bispecific fusion proteins were made based on an antibody specific for HER2 (e.g., an antibody shown in SEQ ID NOs: 79 and 80) and one or more a lipocalin muteins specific for CD137 (e.g., the lipocalin shown in any one sequence of SEQ ID NOs: 21-39). One or more lipocalin muteins were genetically fused to the C- and/or the N-terminus of either the heavy chain or the light chain of a HER2 specific antibody as depicted in FIG. 1A-1D, resulting in the fusion proteins, e.g., SEQ ID NOs: 81 and 80, SEQ ID NOs: 79 and 82, SEQ ID NOs: 83 and 80, and SEQ ID NOs: 79 and 84. An engineered IgG4 backbone with the mutations S228P, F234A, and L235A was used for the HER2 specific antibody as included in the fusion proteins.

### IV. DETAILED DESCRIPTION OF THE DISCLOSURE

CD137 is a costimulatory immune checkpoint and member of the tumor necrosis factor receptor (TNFR) family. It is primarily expressed on activated CD4⁺ and CD8⁺ T cells, activated B cells, and natural killer (NK) cells, and plays an important role in the regulation of the immune response. The clustering of CD137 leads to activation of the receptor and downstream signaling (Yao et al., Nat Rev Drug Discov, 2013, Snell et al., Immunol Rev, 2011). In a T cell pre-stimulated by the T cell receptor (TCR) binding to a cognate major histocompatibility complex (MHC) target, costimulation via CD137 leads to enhanced activation, survival, and proliferation, as well as the production of pro-inflammatory cytokines and an improved capacity to kill.

In line with the mode of CD137 activation, which requires receptor clustering, a monospecific CD137-targeting agent, such as an anti-CD137 antibody, may not be efficient by itself to cluster CD137 and lead to efficient activation. Besides, recent work around TNFR family members illustrates the mechanisms of anti-TNFR antibodies whereby the antibodies interact via their Fc regions with Fc-gamma receptors, engaging activating Fc-gamma receptor-expressing immune cells, and facilitate the subsequent anti-tumor activity (Bulliard et al., Immunol Cell Biol, 2014, Bulliard et al., J Exp Med, 2013). This, therefore, suggests an anti-CD137 antibody may trigger CD137 clustering depending on the abundance of Fc-gamma receptor-positive cells, which are not selectively tumor-localized but distributed throughout the body. Accordingly, the efficacy and target-specificity of anti-CD137 monotherapy may be of concerns. In fact, some anti-CD137 therapeutics under clinical studies, such as urelumab and utomilumab, show disappointing efficacy results with low-dose and/or reveal toxicity at high-dose or effective-dose (Bulliard et al., Immunol Cell Biol, 2014, Bulliard et al., J Exp Med, 2013).

Therefore, there is an unmet need for CD137-targeting therapeutics that are both effective and safe. An ideal CD137-targeting agent should lead to clustering of CD137, and do so in a tumor localized fashion on tumor-infiltrating lymphocytes. As described herein, to obtain such a CD137-targeting agent, bispecific agents may be designed to target CD137 on one end and a differentially expressed tumor target on the other end.

In this respect, HER2 is a clinically-validated target across a broad spectrum of tumor types. Amplification of the HER2 gene and overexpression of its product have been shown to play an important role in the development and progression of various types of cancer including breast, bladder, gastric, gastroesophageal, colorectal, and biliary tract cancer. CD137/HER2 bispecific agents as provided herein are therefore envisioned to promote CD137 clustering by bridging T cells with HER2-positive tumor cells and deliver a costimulatory signal to tumor antigen-specific T cells, providing localized immune activation and leading to tumor destruction.

Furthermore, the combination of checkpoint immunotherapies and immunotherapy and tumor-targeted therapy have been demonstrated to show superiority over monotherapies in some instances (Karachaliou et al., Ann Transl Med, 2017, Ott et al., J Immunother Cancer, 2017). CD137-targeting agents are being explored in combination with other therapies. Although preclinical studies with mouse xenograft models have shown that the anti-CD137 therapy may benefit from combining with checkpoint inhibitors, such as anti-PD-1/PD-L1 antibodies (Dai et al., Clin Cancer Res, 2015, Wei et al., Oncoimmunology, 2014, Chen et al., Cancer Immunol Res, 2015, Kohrt et al., J Clin Invest, 2014, Kohrt et al., J Clin Invest, 2012, Morales-Kastresana et al., J Immunother Cancer, 2013), there remains a need to balance the improvement in efficacy and associated risks including non-targeted immune activation and toxicitiy for real life applications of CD137-targeting combination therapy. Clinically, agonist anti-CD137 antibody urelumab has been studied in combination with nivolumab (anti-PD-1 mAb) presenting discouraging efficacy results and limited clinical activity (Massarelli, 31st Annual Meeting and Associated Programs of the Society for Immunotherapy of Cancer, 2016). Urelumab was also examined in a neoadjuvant trial of cetuximab (anti-EGFR mAb) and revealed the potential to strengthen anti-tumor immunity, albeit only in the presence of NK cells already activated by cetuximab (Srivastava et al., Clin Cancer Res, 2017). Another anti-CD137 antibody, utomilumab is being studied with another PD-1 inhibitor, pembrolizumab. While the phase lb data showed clinical benefit and revealed certain trends in biomarkers, the combination was not sufficient to distinguish between the benefits of utomilumab and the effects of pembrolizumab monotherapy (Tolcher et al., Clin Cancer Res, 2017).

Despite the ongoing developments, there are still needs to identify biomarkers associated with clinical benefit to CD137-targeting therapeutics and optimal combination therapies with suitable modes of actions.

Therefore, in some embodiments, the present disclosure provides combinations comprising a CD137-targeting agent and a PD-1 axis inhibitor. A provided CD137-targeting agent is preferably a CD137/HER2 bispecific agent and accordingly a provided combination will comprise a CD137/HER2 bispecific agent and a PD-1 axis inhibitor.

The present disclosure also provides methods of making one or more combinations comprising a CD137/HER2 bispecific agent with a PD-1 axis inhibitor as well as compositions comprising such combination(s). In some embodiments, the present disclosure includes pharmaceutical compositions, as well as pharmaceutical kits, comprising a CD137/HER2 bispecific agent and a PD-1 axis inhibitor. Provided pharmaceutical compositions may be in solid, liquid, sustained release such as transdermal, transnasal, or depot dosage units and may further include a suitable pharmaceutical carrier. In some embodiments, the present disclosure provides methods of using a combination comprising a CD137/HER2 bispecific agent and a PD-1 axis inhibitor, for the treatment of specific HER2-positive advanced or metastatic solid tumors. The present disclosure also provides methods of using a combination comprising a CD137/HER2 bispecific agent and a PD-1 axis inhibitor, to enhance immune response in an individual having HER2-positive advanced or metastatic solid tumors.

### A. Exemplary Combinations of the Disclosure and Uses and Applications Thereof.

In some embodiments, a provided combination comprises at least a CD137/HER2 bispecific agent and a PD-1 axis inhibitor. In some embodiments, a provided combination comprises at least a CD137/HER2 bispecific agent and a PD-L1 inhibitor. In some embodiments, a provided combination comprises at least a CD137/HER2 bispecific agent that is a CD137/HER2 bispecific fusion protein set forth in SEQ ID NOs: 81 and 80 and a PD-L1 inhibitor that is a PD-L1 antibody set forth in SEQ ID NOs: 76 and 77. In some embodiments, a provided combination comprises at least a CD137/HER2 bispecific agent that is a CD137/HER2 bispecific fusion protein set forth in SEQ ID NOs: 81 and 80 and a PD-1 inhibitor that is a PD-1 antibody set forth in SEQ ID NOs: 72 and 73 or SEQ ID NOs: 74 and 75.

In some embodiments, a provided combination is the combination of a CD137/HER2 bispecific agent and a PD-1 axis inhibitor. In some embodiments, a provided combination is the combination of a CD137/HER2 bispecific agent and a PD-L1 inhibitor. In some embodiments, a provided combination is the combination of a CD137/HER2 bispecific agent and a PD-1 inhibitor. In some embodiments, a provided combination is the combination of a CD137/HER2 bispecific fusion protein set forth in SEQ ID NOs: 81 and 80 and a PD-L1 antibody set forth in SEQ ID NOs: 76 and 77. In some embodiments, a provided combination is the combination a CD137/HER2 bispecific fusion protein set forth in SEQ ID NOs: 81 and 80 and a PD-1 antibody set forth in SEQ ID NOs: 72 and 73. In some embodiments, a provided combination is the combination of a CD137/HER2 bispecific fusion protein set forth in SEQ ID NOs: 81 and 80 and a PD-1 antibody set forth in SEQ ID NOs: 74 and 75.

In some embodiments, a provided combination comprises a CD137/HER2 bispecific agent and a PD-1 axis inhibitor, each in an amount that, in combination with the other, may be able to activate T cells or stimulate T-cell responses. In some embodiments, a provided combination comprises a CD137/HER2 bispecific agent and a PD-1 axis inhibitor, each in an amount that, in combination with the other, may be able to synergistically or additively activate T cells or stimulate T-cell responses. In some embodiments, a provided combination leads to T-cell activation with a comparable or better potency and/or efficacy as compared to a CD137/HER2 bispecific agent (e.g., SEQ ID NOs: 81 and 80, SEQ ID NOs: 79 and 82, SEQ ID NOs: 82 and 80, and SEQ ID NOs: 79 and 84) and/or a PD-1 axis inhibitor (e.g. SEQ ID NOs: 72 and 73, SEQ ID NOs: 74 and 75, and SEQ ID NOs: 76 and 77) comprised in a particular combination. The stimulated T-cell response or T-cell activation may be measured, for example, in a functional T-cell activation assay as essentially described in Examples 1-3.

In some embodiments, a provided combination comprises a CD137/HER2 bispecific agent and a PD-1 axis inhibitor, each in an amount that, in combination with the other, may be able to induce increased IL-2 secretion relative to the bispecific agent or inhibitor alone. In some embodiments, a provided combination comprises a CD137/HER2 bispecific agent and a PD-1 axis inhibitor, each in an amount that, in combination with the other, may be able to synergistically or additively induce increased IL-2 secretion. In some preferred embodiments, provided combinations may be able to induce a concentration-dependent IL-2 secretion. In some embodiments, provided combinations may lead to increased IL-2 secretion with a comparable or better efficiency as compared to to a CD137/HER2 bispecific agent (e.g. SEQ ID NOs: 81 and 80, SEQ ID NOs: 79 and 82, SEQ ID NOs: 82 and 80, and SEQ ID NOs: 79 and 84) and/or a PD-1 axis inhibitor (e.g. SEQ ID NOs: 72 and 73, SEQ ID NOs: 74 and 75, and SEQ ID NOs: 76 and 77) alone. IL-2 secretion may be measured, for example, in a functional T-cell activation assay as essentially described in Examples 1-3.

In some embodiments, a provided combination comprises a CD137/HER2 bispecific agent and a PD-1 axis inhibitor, each in an amount that, in combination with the other, may be able to stimulate T-cell responses in the presence of tumor cells and/or in a tumor microenvironment. In some particular embodiments, a provided fusion protein may be able to stimulate T-cell responses in the presence of HER2-positive tumor cells. The T-cell activation by provided combinations in the presence of tumor cells and/or in a tumor microenvironment may be assessed, for example, in a functional T-cell activation assay essentially described in Examples 1-3.

In some embodiments, a provided combination may be able to activate T cells or stimulate T-cell responses in a HER2 dependent manner. In some embodiments, provided combinations may lead to local induction of the IL-2 production by T-cells in the microenvironment in HER2-positive cancer. The HER2 dependent activation of T-cell by provided combinations may be determined, for example, in a functional T-cell activation assay essentially described in Example 3, in which a combination of a CD137/HER2 bispecific agent and a PD-1 axis inhibitor was tested in the presence of tumor cells expressing different levels of HER2 and demonstrated the ability to activate T cells differently depending on the HER2 expression level.

In some embodiments, a provided combination comprises a CD137/HER2 bispecific agent and a PD-1 axis inhibitor, each in an amount that, in combination with the other, exerts additive anti-tumor effects. In some embodiments, a provided combination comprises a CD137/HER2 bispecific agent and a PD-1 axis inhibitor, each in an amount that, in combination with the other, exerts synergistic anti-tumor effects. Provided combinations, thereof, in some embodiments, when administered, allow lower doses with improved efficacy and a wider safety window, as compared to a CD137/HER2 bispecific agent (e.g., SEQ ID NOs: 81 and 80, SEQ ID NOs: 79 and 82, SEQ ID NOs: 83 and 80, SEQ ID NOs: 79 and 84) alone and/or a PD-1 axis inhibitor (e.g. SEQ ID NOs: 72 and 73, SEQ ID NOs: 74 and 75, SEQ ID NOs: 76 and 77) alone.

In some embodiments, a provided combination comprises a CD137/HER2 bispecific agent and a PD-1 axis inhibitor, each in an amount that, in combination with the other, may be able to induce increased secretion of IL-2. In some embodiments, a provided combination comprises a CD137/HER2 bispecific agent and a PD-1 axis inhibitor, each in an amount that, in combination with the other, may be able to induce increased secretion of IFN-gamma.

In some embodiments, a provided combination comprises a CD137/HER2 bispecific agent and a PD-1 axis inhibitor, each in an amount that, in combination with the other, may be able to stimulate CD4⁺ T cell proliferation and/or activation, preferably in a tumor microenvironment.

In some embodiments, a provided combination comprises a CD137/HER2 bispecific agent and a PD-1 axis inhibitor, each in an amount that, in combination with the other, may be able to stimulate CD8⁺ T cell proliferation and/or activation, preferably in a tumor microenvironment.

In some embodiments, a provided combination comprises a CD137/HER2 bispecific agent and a PD-1 axis inhibitor, each in an amount that, in combination with the other, may be able to induce the expansion of tumor-infiltrating lymphocytes.

In some embodiments, a provided combination comprises a CD137/HER2 bispecific agent and a PD-1 axis inhibitor, each in an amount that, in combination with the other, may be able to activate NK cells and increase ADCC, preferably in a tumor microenvironment.

In some embodiments, a provided combination comprises a CD137/HER2 bispecific agent and a PD-1 axis inhibitor, each in an amount that, in combination with the other, may be able to induce changes in biomarker levels in a subject. In some embodiments, a provided combination may decrease the level of a biomarker in a subject. In some embodiments, a provided combination may increase the level of a biomarker in a subject. The biomarker may be, for example, CD4, CD8, PD-L1, Ki67, CD137, HER2, IL-8, and FoxP3.

In some embodiments, a provided combination comprises a CD137/HER2 bispecific agent and a PD-1 axis inhibitor, each in an amount that, in combination with the other, may be able to reduce the tumor size in a subject.

In some embodiments, a provided combination comprises a CD137/HER2 bispecific agent and a PD-1 axis inhibitor, each in an amount that, in combination with the other, may be able to suppress tumor growth in a subject.

In some embodiments, a provided combination comprises a CD137/HER2 bispecific agent and a PD-1 axis inhibitor, each in an amount that, in combination with the other, may be able to suppress tumor metastasis in a subject.

In some embodiments, a provided combination comprises a CD137/HER2 bispecific agent and a PD-1 axis inhibitor, each in an amount that, in combination with the other, may be able to delay tumor recurrence in a subject.

In some embodiments, a provided combination comprises a CD137/HER2 bispecific agent and a PD-1 axis inhibitor, each in an amount that, in combination with the other, may be able to improve overall survival for a subject.

In some embodiments, provided combinations may be administered to a subject, e.g., a mammal such as a human. In some embodiments, a subject administered with provided combinations may be confirmed diagnosis of previously treated advanced and/or metastatic HER2-positive tumor.

In some embodiments, components of a provided combination may be administered simultaneously or sequentially with respect to any other component of the combination. For example, a CD137/HER2 bispecific agent and a PD-1 axis inhibitor may be administered simultaneously with one another or sequentially with respect to each other.

In some embodiments, components of a provided combination may be administered as adjuvant to any other component of the combination, i.e., one component of a provided combination may be administered after a subject receives the treatment of another component. For example, a CD137/HER2 bispecific agent may be administered after a subject receives the treatment of a PD-1 axis inhibitor or a PD-1 axis inhibitor may be administered after a subject receives the treatment of a CD137/HER2 bispecific agent.

In some embodiments, when the components of a provided combination are administered simultaneously, they can be administered in a single formulation or in distinct formulations. In some embodiments, when the components of a provided combination are administered as distinct formulations, whether simultaneously or sequentially, the components may be administered at a single site or at separate sites and using the same route or different routes. In some embodiments, when the components of a provided combination are administered sequentially, the time between administration of the components may be determined using certain factors such as the length of time a particular component persists, either systemically or at the administration site; or the length of time that the cellular effects of the component persist, either systemically or at the administration site.

In some embodiments, combinations of the disclosure, or compositions comprising provided combinations are envisaged to be used as anti-tumor agents, anti-infection agents, and/or immune modulators. For example, in some embodiments, provided combinations may be used for increasing IL-2 secretion. In some embodiments, provided combinations may be used for increasing INF-gamma secretion. In some embodiments, provided combinations may be used for increasing tumor-infiltrating lymphocytes. In some embodiments, provided combinations may be used for activating NK cells and increasing ADCC. In some embodiments, provided combinations may be used for inducing lymphocyte activation and/or proliferation. In some embodiments, provided combinations may be used for enhancing immune functions. In some embodiments, provided combinations may be used for inducing CD137 clustering and activation on T cells and directing such T cells to tumor cells, preferably HER2-positive tumor cells. In some embodiments, provided combinations may be used for inducing a lymphocyte response in tumor microenvironment. In some embodiments, provided combinations may be used for providing anti-tumor effects. In some embodiments, provided combinations may be used for inducing changes in the level of a biomarker, such as, CD4, CD8, PD-L1, Ki67, CD137, HER2, IL-8, and FoxP3. In some embodiments, provided combinations may be used for treating and/or delaying progression of a cancer.

In some embodiments, provided combinations may be used for treating HER2-expressing tumors. In some embodiments, provided combinations may be used for treating HER2-positive tumors. In some embodiments, provided combinations may be used for treating HER2-equivocal tumors. In some embodiments, provided combinations may be used for treating HER2-negative tumors. In some embodiments, provided combinations may be used for treating HER2-expressing tumors. The HER2 expressing status of a tumor may, for example, be analyzed at the protein level by immunohistochemistry (IHC) or other methods known in the art. In some embodiments, provided combinations may be used for treating HER2-positive advanced or metastatic solid tumors.

In some embodiments, provided combinations may be used for treating PD-L1-positive tumors.

In some embodiments, each component of a provided combination may, by itself, possess anti-tumor, anti-infective, and/or immunostimulatory activity. In some embodiments, combinations of components, such as a combination of a CD137/HER2 bispecific agent and a PD-1 axis inhibitor, may provide greater anti-tumor, anti-infective, and/or immunostimulatory activity any one single component may provide. In some embodiments, combinations of components, may provide additive or synergistic anti-tumor, anti-infective, and/or immunostimulatory activity.

In some embodiments, provided combinations may have favorable pharmacokinetic properties to permit a dosing schedule of about twice a week, of about once a week, of about once every ten days, of about once every two weeks, of about once every three weeks, of about once every four weeks, of about once every five weeks, of about once every month, of about once every six weeks, of about once every seven weeks, of about once every eight weeks, or of about once every two months. In some embodiments, one component of a provided combination may, by itself, have favorable pharmacokinetic properties.

In some embodiments, one or more components of a provided combination are administered with a frequency of about twice a week, of about once a week, of about once every ten days, of about once every two weeks, of about once every three weeks, of about once every four weeks, of about once every five weeks, of about once every month, of about once every six weeks, of about once every seven weeks, of about once every eight weeks, or of about once every two months.

In some embodiments, a combination is provided in the form of a kit of parts. In some embodiments, a kit of part comprises a pharmaceutical composition comprising a CD137/HER2 bispecific agent and a pharmaceutical composition comprising a PD-1 axis inhibitor. In some embodiments, a pharmaceutical composition comprising a CD137/HER2 bispecific agent and a pharmaceutical composition comprising a PD-1 axis inhibitor are provided in at least two separate unit dosage forms. In some embodiments, pharmaceutical compositions comprised in a kit of parts are provided in a unit dosage that corresponds to the dose to be administered to a subject per single administration. As an illustrative example, a CD137/HER2 bispecific agent is administered at a unit dosage of about 0.05 mg/kg, 0.15 mg/kg, 0.5 mg/kg, 1.0 mg/kg, 2.5 mg/kg, 5.0 mg/kg, or 8.0 mg/kg and a PD-L1 axis inhibitor is administered at a unit dosage of about 1200 mg.

### B. Exemplary CD137/HER2 Bispecific Agents.

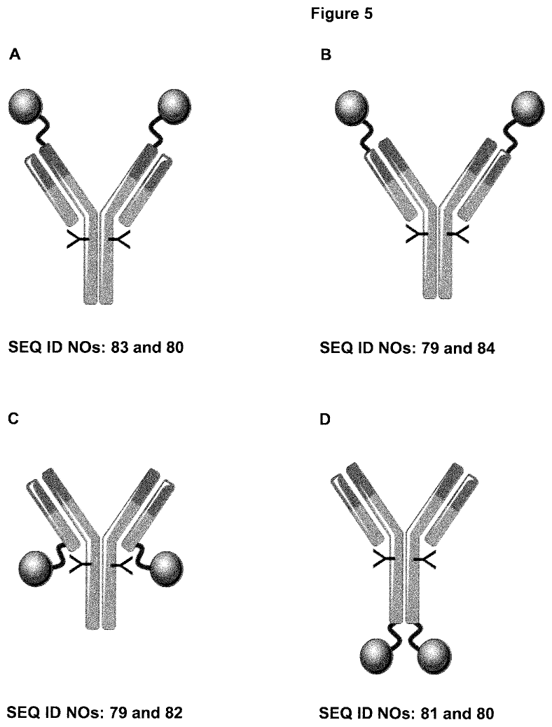

In some embodiments, a provided combination comprises a CD137/HER2 bispecific agent. In some embodiments, a CD137/HER2 bispecific agent of the disclosure may be a CD137/HER2 bispecific fusion protein containing at least two subunits in any order: (1) a first subunit that comprises a full-length immunoglobulin or an antigen-binding domain thereof specific for HER2, and (2) a second subunit that comprises a lipocalin mutein specific for CD137 (FIG. 5). In some embodiments, a provided CD137/HER2 bispecific agent may be used in a method provided herein.

In some embodiments, a provided CD137/HER2 bispecific fusion protein also may contain at least one additional subunit, for example, a third subunit. For instance, a fusion protein may contain a third subunit specific for CD137. In some embodiments, a provided CD137/HER2 bispecific fusion protein may comprise one or more additional subunits (e.g., a fourth, fifth, or sixth subunit).

In some embodiments, at least one subunit may be fused at its N-terminus and/or its C-terminus to another subunit.

In some embodiments, at least one subunit can be fused to another subunit via a linker. In some further embodiments, a linker is a peptide linker, for example, an unstructured glycine-serine (GS) linker, a glycosylated GS linker, or a proline-alanine-serine polymer (PAS) linker. In some embodiments, a GS linker is a (Gly₄Ser)₃ linker ((G₄S)₃) as shown in SEQ ID NO: 4. Other exemplary linkers are shown in SEQ ID NOs: 5-14. In some embodiments, a peptide linker may have from 1 to 50 amino acids, such as 1, 2, 3, 4, 5, 10, 11, 12, 13, 14, 15, 16, 17 18, 19, 20, 25, 30, 35, 40, 45 or 50 amino acids. For example, in some embodiments, when a first subunit comprises a full-length immunoglobulin, a second subunit may be linked via a peptide linker between the N-terminus of the second subunit and the C-terminus of a heavy chain constant region (CH) of said immunoglobulin. In some further embodiments, a third subunit may be linked via a peptide linker between the N-terminus of the third subunit and the C-terminus of a light chain constant region (CL) of said immunoglobulin.

In some embodiments, a lipocalin mutein subunit may be fused to an immunoglobulin subunit of a provided CD137/HER2 bispecific fusion protein. In some embodiments, a lipocalin mutein subunit may be fused at its N-terminus and/or its C-terminus to an immunoglobulin subunit at the C-terminus of the immunoglobulin heavy chain domain (HC), the N-terminus of the HC, the C-terminus of the immunoglobulin light chain (LC), and/or the N-terminus of the LC (FIG. 5). For example, in some embodiments, a lipocalin mutein may be linked, via a peptide linker, at its N-terminus to each of the HC of an immunoglobulin (FIG. 5D).

In some embodiments, with respect to a CD137/HER2 bispecific fusion protein of the disclosure, wherein at least one subunit may be or comprise a full-length immunoglobulin, the Fc function of the Fc region of the full-length immunoglobulin to Fc receptor-positive cell may be preserved at the same time while the fusion protein is simultaneously engaging CD137 and HER2.

In some embodiments, wherein at least one subunit of a provided CD137/HER2 bispecific fusion protein may be or comprise a full-length immunoglobulin, the Fc function of the Fc region of the full-length immunoglobulin to Fc receptor-positive cell may be reduced or fully suppressed by protein engineering while the fusion protein is simultaneously engaging CD137 and PD-L1. In some embodiments, this may be achieved, for example, by switching from the IgG1 backbone to IgG4, as IgG4 is known to display reduced Fc-gamma receptor interactions compared to IgG1. In some embodiments, to further reduce the residual binding to Fc-gamma receptors, mutations may be introduced into the IgG4 backbone such as F234A and L235A. In some embodiments, an S228P mutation may also be introduced into the IgG4 backbone to minimize the exchange of IgG4 half-antibody (Silva et al., J Biol Chem, 2015). In some embodiments, F234A and L235A mutations may be introduced for decreased ADCC and ADCP (Glaesner et al., Diabetes Metab Res Rev, 2010) and/or M428L and N434S mutations or M252Y, S254T, and T256E mutations for extended serum half-life (Dall'Acqua et al., J Biol Chem, 2006, Zalevsky et al., Nat Biotechnol, 2010). In some embodiments, an additional N297A mutation may be present in the immunoglobulin heavy chain of a provided CD137/HER2 bispecific fusion protein in order to remove the natural glycosylation motif.

In some embodiments, the Fc portion of an immunoglobulin included in a CD137/HER2 bispecific fusion protein of the disclosure may contribute to maintaining the serum levels of the fusion protein. For example, when the Fc portion binds to Fc receptors on endothelial cells and phagocytes, the fusion protein may become internalized and recycled back to the bloodstream, enhancing its half-life within the body.

In some embodiments, with respect to a provided CD137/HER2 bispecific fusion protein, a first subunit may be or comprise a full-length immunoglobulin or an antigen-binding domain thereof specific for HER2. In some embodiments, an immunoglobulin is a monoclonal antibody against HER2.

In some embodiments, a provided HER2 antibody or antigen-binding domain thereof may comprise the three heavy chain CDRs of SEQ ID NO: 40, SEQ ID NO: 41, and SEQ ID NO: 42, and/or the three light chain CDRs of SEQ ID NO: 43, SEQ ID NO: 44, SEQ ID NO: 45.

In some embodiment, a provided HER2 antibody or antigen-binding domain thereof may have a heavy chain variable region (HCVR) shown in SEQ ID NO: 64, and/or a light chain variable region (LCVR) shown in SEQ ID NO: 65.

In some embodiments, a provided HER2 antibody or antigen-binding domain thereof may have a heavy chain that is any one of SEQ ID NO: 78 or 79, and/or a light chain shown in SEQ ID NO: 80.

In some embodiments, a provided HER2 antibody or antigen-binding domain thereof may have a HCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 64, and/or a LCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 65. In other embodiments, a provided HER2 antibody or antigen-binding domain thereof may have a heavy chain with at least 70%, at least 75%, at least 80%, at least 85%, at least 90%, at least 92%, at least 95%, at least 97%, at least 98%, at least 99%, or even higher sequence identity to an amino acid sequence selected from the group consisting of SEQ ID NOs: 78-79, and/or a light chain with at least 70%, at least 75%, at least 80%, at least 85%, at least 90%, at least 92%, at least 95%, at least 97%, at least 98%, at least 99%, or even higher sequence identity to the amino acid sequence of SEQ ID NO: 80.

In some embodiments, a provided HER2 antibody is trastuzumab. In some embodiments, a provided HER2 antibody is trastuzumab with an IgG4 backbone.

In some embodiments, with respect to a provided CD137/HER2 bispecific fusion protein, a second subunit may be or comprise a lipocalin mutein specific for CD137. In some embodiments, a provided lipocalin mutein may be or comprise a mutein of mature human neutrophil gelatinase-associated lipocalin (hNGAL). A mutein of mature hNGAL may be designated herein as an “hNGAL mutein”.

In some embodiments, a provided CD137-binding hNGAL mutein may bind human CD137 with high affinity and be capable of costimulating human T cells when immobilized on a plastic dish together with an anti-CD3 antibody. In some embodiments, a provided CD137-binding hNGAL mutein may comprise an amino acid sequence selected from the group consisting of SEQ ID NOs: 21-39 or of a fragment or variant thereof. In some embodiments, a provided CD137-binding hNGAL mutein may comprise the amino acid sequence of SEQ ID NOs: 22 or of a fragment or variant thereof. In some embodiments, the amino acid sequence of a provided CD137-binding hNGAL mutein may have a high sequence identity, such as at least 70%, at least 75%, at least 80%, at least 82%, at least 85%, at least 87%, at least 90%, at least 95%, at least 98%, at least 99%, or higher identity, to a sequence selected from the group consisting of SEQ ID NOs: 21-39. In some embodiments, the amino acid sequence of a provided CD137-binding hNGAL mutein may have a high sequence identity, such as at least 70%, at least 75%, at least 80%, at least 82%, at least 85%, at least 87%, at least 90%, at least 95%, at least 98%, at least 99%, or higher identity, to the sequence of SEQ ID NOs: 22.

In some embodiments, a provided CD137/HER2 bispecific fusion protein is generated by genetic fusion of a CD137-specific hNGAL mutein to a trastuzumab IgG4 variant, connected by a flexible, non-immunogenic linker.

In some embodiments, a provided CD137/HER2 bispecific fusion protein is capable of engaging HER2 and CD137 simultaneously. In some embodiments, a provided fusion protein may be able to activate CD137 signaling in a HER2-dependent manner. In some embodiments, a provided fusion protein may be able to activate CD137 signaling in HER2-positive tumor microenvironment. In some embodiments, a provided fusion protein may be able to activate costimulate T cell responses and/or enhance T cell function in HER2-positive tumor microenvironment.

In some embodiments, a provided CD137/HER2 bispecific fusion protein may comprise the amino acid sequences selected from the group consisting of SEQ ID NOs: 81 and 80, SEQ ID NOs: 79 and 82, SEQ ID NOs: 83 and 80, SEQ ID NOs: 79 and 84.

In some embodiments, a provided CD137/HER2 bispecific fusion protein may comprise the amino acid sequences having at least 70%, at least 75%, at least 80%, at least 85%, at least 90%, at least 92%, at least 95%, at least 97%, at least 98%, at least 99%, or higher sequence identity to the amino acid sequences shown in SEQ ID NOs:81 and 80, SEQ ID NOs: 79 and 82, SEQ ID NOs: 83 and 80, SEQ ID NOs: 79 and 84. In some embodiments, where the bispecific fusion protein comprise more than one amino acid chain, a given value for the sequence identity relates to the average sequence identity normalized by the number of amino acid residues in both amino acid chains. For example, if a bispecific fusion protein consists of amino acid chain A having 100 amino acids and amino acid chain B having 50 amino acids, and another bispecific fusion protein consists of amino acid chain A′ having 100 amino acids 80% sequence identity to amino acid chain A and amino acid chain B′ having 50 amino acids and 95% sequence identity to amino acid chain B′, the average sequence identity between both fusion proteins will be (100/(100+50))×80%+(50/(100+50))×95%=85% sequence identity. In some preferred embodiments, where the bispecific fusion protein comprise more than one amino acid chain, a given value for the sequence identity means that a protein of interest comprises an amino acid sequence that has at least the given value of sequence identity to one chain of the bispecific fusion protein and comprises an amino acid sequence that has at least the given value of sequence identity to the other chain of the bispecific fusion protein.

### C. Exemplary PD-1 Axis Inhibitors.

In some embodiments, a provided combination comprises a component of a PD-1 axis inhibitor. In some embodiments, a PD-1 axis inhibitor of the disclosure may be a PD-1 antagonist, a PD-L1 antagonist, or a PD-L2 antagonist. In some embodiments, a provided PD-1 axis inhibitor is an anti-PD-1 antibody, anti-PD-L1 antibody, or an anti-PD-L2 antibody. In some embodiments, a provided PD-1 axis inhibitor may be used in a method provided herein.

Various PD-1 axis inhibitors are known in the art. Pembrolizumab, also known as MK-3475, Merck 3475, lambrolizumab, SCH-900475, and KEYTRUDA®, is a humanized IgG4 monoclonal antibody that binds to and blocks PD-1. It is used via intravenous infusion to treat inoperable or metastatic melanoma, metastatic non-small cell lung cancer (NSCLC) in certain situations, as a second-line treatment for head and neck squamous cell carcinoma (HNSCC), after platinum-based chemotherapy, and for the treatment of adult and pediatric patients with refractory classic Hodgkin's lymphoma (cHL). Nivolumab, also known as MDX-1106-04, MDX-1106, ONO-4538, BMS-936558, and OPDIVO®, is a humanized IgG4 anti-PD-1 monoclonal antibody which blocks PD-L1 from binding to PD-1. It is used as a first line treatment for inoperable or metastatic melanoma in combination with ipilimumab if the cancer does not have a mutation in BRAF, as a second-line treatment following treatment with ipilimumab and if the cancer has a mutation in BRAF, with a BRAF inhibitor, as a second-line treatment for squamous non-small cell lung cancer, and as a second-line treatment for renal cell carcinoma. Atezolizumab, also known as MPDL3280A or Tecentriq®, is a human IgG1 monoclonal antibody engineered to eliminate Fc-effector function. It targets human PD-L1 and inhibits its interaction with PD-1. Atezolizumab is indicated for the treatment of urothelial carcinoma, a common type of bladder cancer, and is under investigation in other tumor types.

### C-1. Exemplary Anti-PD-L1 Antibodies as PD-1 Axis Inhibitors

In some embodiments, a provided PD-1 axis inhibitor is an anti-PD-L1 antibody or antigen-binding domain thereof. In some embodiments, a provided combination comprises an anti-PD-L1 antibody or antigen-binding domain thereof.

Illustrative examples of anti-PD-L1 antibodies of the disclosure may comprise an antigen-binding regions which cross-blocks or binds to the same epitope as an anti-PD-L1 antibody comprising the VH and VL regions of a previously known antibody, such as atezolizumab (also known as MPDL3280A or RG7446, trade name Tecentrie), avelumab (also known as MSB0010718C, trade name Bavencie), durvalumab (previously known as MED14736, trade name Imfinzi®), BMS-936559 (also known as MDX-1105), and anti-PD-L1 antibodies disclosed in PCT International Application Publication Nos. WO 2010/077634, WO 2007/005874, WO 2010/089411, WO 2011/066389, WO 2013/079174, WO 2015/048520, WO 2016/061142, WO 2016/111645, WO 2015/061668, WO 2016/007235, and WO 2017/148424. A provided anti-PD-L1 antibody also may be or comprises an antigen-binding region or the VH and VL regions or of any one of the above-mentioned anti-PD-L1 antibodies.

In some embodiments, a provided anti-PD-L1 antibody or antigen-binding domain thereof may comprise an antigen-binding region, such as any one of the three heavy chain complementarity-determining regions (CDRs) (HCDR1, HCDR2 and HCDR3) and the three light chain CDRs (LCDR1, LCDR2 and LCDR3) from an antibody selected from the group consisting of atezolizumab, avelumab, durvalumab, and anti-PD-L1 antibodies as described in WO2017148424.

In some embodiments, a provided PD-L1 antibody or antigen-binding domain thereof may comprise the three heavy chain CDRs of SEQ ID NO: 58, SEQ ID NO: 59, and SEQ ID NO: 60, and/or the three light chain CDRs of SEQ ID NO: 61, SEQ ID NO: 62, SEQ ID NO: 63.

In some embodiments, a provided anti-PD-L1 antibody or antigen-binding domain thereof may have a heavy chain variable region (HCVR) shown in SEQ ID NO: 70, and/or a light chain variable region (LCVR) shown in SEQ ID NO: 71.

In some embodiments, a provided PD-L1 antibody or antigen-binding domain thereof may have a heavy chain shown in SEQ ID NO: 76, and/or a light chain shown in SEQ ID NO: 77.

In some embodiments, a provided PD-L1 antibody or antigen-binding domain thereof may have a HCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 70, and/or a LCVR with at least 70%, at least 75%, at least 80%, at least 85%, at least 90%, at least 92%, at least 95%, at least 97%, at least 98%, at least 99%, or even higher sequence identity to an amino acid sequence shown in SEQ ID NO: 71. In some embodiments, a provided PD-L1 antibody or antigen-binding domain thereof may have a heavy chain with at least 70%, at least 75%, at least 80%, at least 85%, at least 90%, at least 92%, at least 95%, at least 97%, at least 98%, at least 99%, or even higher sequence identity to an amino acid sequence of SEQ ID NOs: 76, and/or a light chain with at least 70%, at least 75%, at least 80%, at least 85%, at least 90%, at least 92%, at least 95%, at least 97%, at least 98%, at least 99%, or even higher sequence identity to the amino acid sequence of SEQ ID NO: 77.

In some embodiments, an anti-PD-L1 antibody of the disclosure is atezolizumab. In some embodiments, an anti-PD-L1 antibody of the disclosure is durvalumab or avelumab.

### C-2. Exemplary Anti-PD-1 Antibodies as the PD-1 Axis Inhibitor

In some embodiments, a provided PD-1 axis inhibitor is an anti-PD-1 antibody or antigen-binding domain thereof. In some embodiments, a provided combination comprises an anti-PD-1 antibody or antigen-binding domain thereof.

Illustrative examples of anti-PD-1 antibodies of the disclosure may comprise an antigen-binding region which cross-blocks or binds to the same epitope as an anti-PD-1 antibody comprising the VH and VL regions of a previously known antibody, such as nivolumab (also known as ONO-4538, BMS-936558, or MDX1106, marketed as Opdivo®), pembrolizumab (also referred to as lambrolizumab or MK03475, trade name Keytruda®), PDR001, tislelizumab (BGB-A317), cemiplimab (REGN281), MEDI0680 (formerly AMP-514), pidilizumab (CT-011), ENUM-388D4 (including the D4-1, D4-2 and D4-3 variants), ENUM-244C8 (including each of its variants as well), and anti-PD-1 antibodies disclosed in the U.S. Patent Application Publication Nos. US 2003/0039653, US 2004/0213795, US 2006/0110383, US 2007/0065427, US 2007/0122378, US 2009/0217401, US 2011/0008369, and US2015/0203579 and PCT International Application Publication Nos. WO 2003/099196, WO 2006/121168, WO 2007/005874, WO 2008/156712, WO 2009114335, WO 2010/027423, WO2 011/110604, WO 2012/145493, WO 2013/014668, WO 2014/194302, WO 2015/035606, and WO 2016/106159. A provided anti-PD-1 antibody also may be or comprises an antigen-binding region or the VH and VL regions or of any one of the above-mentioned anti-PD-1 antibodies.

In some embodiments, a provided anti-PD-1 antibody or antigen-binding domain thereof may comprise an antigen-binding region, such as any one of the three heavy chain CDRs (HCDR1, HCDR2 and HCDR3) and the three light chain CDRs (LCDR1, LCDR2 and LCDR3) from an antibody selected from the group consisting of nivolumab, pembrolizumab, PDR001, MEDI0680, pidilizumab, ENUM-388D4, and ENUM-244C8.

In some embodiments, a provided PD-1 antibody or antigen-binding domain thereof may comprise the three heavy chain CDRs of SEQ ID NO: 46, SEQ ID NO: 47, and SEQ ID NO: 48, and/or the three light chain CDRs of SEQ ID NO: 49, SEQ ID NO: 50, SEQ ID NO: 51. In some embodiments, a provided PD-1 antibody or antigen-binding domain thereof may comprise the three heavy chain CDRs of SEQ ID NO: 52, SEQ ID NO: 53, and SEQ ID NO: 54, and/or the three light chain CDRs of SEQ ID NO: 55, SEQ ID NO: 56, SEQ ID NO: 57.

In some embodiments, a provided anti-PD-1 antibody or antigen-binding domain thereof may have a HCVR of any one of SEQ ID NOs: 66 and 68, and/or a light chain variable LCVR of any one of SEQ ID NOs: 67 and 69.

In some embodiments, a provided PD-1 antibody or antigen-binding domain thereof may have a heavy chain of any one of SEQ ID NOs: 72 and 74, and/or a light chain of any one of SEQ ID NOs: 73 and 75.

In some embodiments, the heavy chain and light chain pair of a provided PD-1 antibody or antigen-binding domain thereof comprise the HCVR and LCVR, respectively, as follows: SEQ ID NOs: 66 and 67 or SEQ ID NOs: 68 and 69.

In some embodiments, the heavy chain and light chain pair of a provided PD-1 antibody are or comprise the amino acid sequences as shown in SEQ ID NOs: 72 and 73 or SEQ ID NO: 74 and 75.

In some embodiments, a provided PD-1 antibody or antigen-binding domain thereof may have a HCVR with at least 70%, at least 75%, at least 80%, at least 85%, at least 90%, at least 92%, at least 95%, at least 97%, at least 98%, at least 99%, or even higher sequence identity to an amino acid sequence shown in any one of SEQ ID NOs: 66 and 68, and/or a LCVR with at least 70%, at least 75%, at least 80%, at least 85%, at least 90%, at least 92%, at least 95%, at least 97%, at least 98%, at least 99%, or even higher sequence identity to an amino acid sequence shown in any one of SEQ ID NOs: 67 and 69. In some embodiments, a provided PD-1 antibody or antigen-binding domain thereof may have a heavy chain with at least 70%, at least 75%, at least 80%, at least 85%, at least 90%, at least 92%, at least 95%, at least 97%, at least 98%, at least 99%, or even higher sequence identity to an amino acid sequence of any one of SEQ ID NOs: 72 and 74, and/or a light chain with at least 70%, at least 75%, at least 80%, at least 85%, at least 90%, at least 92%, at least 95%, at least 97%, at least 98%, at least 99%, or even higher sequence identity to the amino acid sequence of any one of SEQ ID NO: 73 and 75.

In some embodiments, an anti-PD-1 antibody of the disclosure is nivolumab or pembrolizumab. In some embodiments, an anti-PD-1 antibody of the disclosure may be tislelizumab or cemiplimab.

### D. Exemplary Methods of the Disclosure

In some embodiments, the present disclosure provides prophylactic and/or therapeutic methods, comprising administering to a subject an effective amount of a combination comprising a CD137/HER2 bispecific agent and a PD-1 axis inhibitor.

In some embodiments, the present disclosure provides methods for increasing IL-2 secretion, preferably in a tumor microenvironment. The method may comprise administering to a subject a combination comprising a CD137/HER2 bispecific agent and a PD-1 axis inhibitor, each in an amount that, when in combination with the other, is effective to increase IL-2 secretion, preferably in a tumor microenvironment, relative to the bispecific agent or the inhibitor alone. In some embodiments, a provided method may elicit a synergistic increase in IL-2 secretion. In some embodiments, a provided method may elicit an additive increase in IL-2 secretion.

In some embodiments, the present disclosure provides methods for increasing IFN-gamma secretion, preferably in a tumor microenvironment. The method may comprise administering to a subject a combination comprising a CD137/HER2 bispecific agent and a PD-1 axis inhibitor, each in an amount that, when in combination with the other, is effective to increase IFN-gamma secretion, preferably in a tumor microenvironment, relative to the bispecific agent or the inhibitor alone. In some embodiments, a provided method may elicit a synergistic increase in IFN-gamma secretion. In some embodiments, a provided method may elicit an additive increase in IFN-gamma secretion.

In some embodiments, the present disclosure provides methods for inducing T lymphocyte activation and/or proliferation. The method may comprise administering to a subject a combination comprising a CD137/HER2 bispecific agent and a PD-1 axis inhibitor, each in an amount that, when in combination with the other, is effective to induce T lymphocyte activation and/or proliferation. In some embodiments, a provided method may elicit a synergistic T lymphocyte activation and/or proliferation. In some embodiments, a provided method may elicit an additive T lymphocyte activation and/or proliferation.

In some embodiments, the present disclosure provides methods for enhancing immune functions. The method may comprise administering to a subject a combination comprising a CD137/HER2 bispecific agent and a PD-1 axis inhibitor, each in an amount that, when in combination with the other, is effective to induce enhanced immune functions. In some embodiments, a provided method may elicit a synergistic enhancement of immune function. In some embodiments, a provided method may elicit an additive enhancement of immune function.

In some embodiments, the present disclosure provides methods for increasing CD4⁺ T cells, preferably in a tumor microenvironment. In some embodiments, the present disclosure provides methods for increasing CD8⁺ T cells, preferably in a tumor microenvironment. In some embodiments, the present disclosure provides methods for activating NK cells and increasing ADCC, preferably in a tumor microenvironment. In some embodiments, the present disclosure provides methods for monitoring and changing tumor biomarker levels, such as CD4, CD8, PD-L1, Ki67, CD137, HER2, and IL-8 levels. Provided methods may comprise administering to a subject a combination comprising a CD137/HER2 bispecific agent and a PD-1 axis inhibitor, each in an amount that, when in combination with the other, is effective to induce expansion of CD4⁺ T cells preferably in a tumor microenvironment, expansion of CD8⁺ T cells preferably in a tumor microenvironment, activation of NK cells and increased ADCC preferably in a tumor microenvironment, and/or changes in tumor biomarker levels.

In some embodiments, the present disclosure provides methods for inducing CD137 clustering and activation on T cells and directing such T cells to tumor cells, preferably HER2-positive tumor cells. The method may comprise administering to a subject a combination comprising a CD137/HER2 bispecific agent and a PD-1 axis inhibitor, each in an amount that, when in combination with the other, is effective to induce CD137 clustering and activation on T cells and directing such T cells to tumor cells, preferably HER2-positive tumor cells.

In some embodiments, the present disclosure provides methods for inducing a localized lymphocyte response in the vicinity of tumor cells, preferably HER2-positive tumor cells. The method may comprise administering to a subject a combination comprising a CD137/HER2 bispecific agent and a PD-1 axis inhibitor, each in an amount that, when in combination with the other, is effective to induce a localized lymphocyte response in the vicinity of tumor cells, preferably HER2-positive tumor cells.

In some embodiments, the present disclosure provides methods for treating or delaying cancer progression. In some embodiments, the present disclosure provides methods for providing anti-tumor effects, such as reduction of tumor size, suppression of tumor growth, suppression of metastasis, delay of recurrence, and improved overall survival. Provided methods may comprise administering to a subject a combination comprising a CD137/HER2 bispecific agent and a PD-1 axis inhibitor, each in an amount that, when in combination with the other, exerts anti-tumor effects.

In some embodiments, the present disclosure provides methods for treating HER2-positive advanced or metastatic solid tumors. In some embodiments, the present disclosure provides methods for treating PD-L1-positive tumors. Provided methods may comprise administering to a subject a combination comprising a CD137/HER2 bispecific agent and a PD-1 axis inhibitor, each in an amount that, when in combination with the other, is effective to exert anti-tumor effects. In some embodiments, a provided method may elicit a synergistic anti-tumor effect. In some embodiments, a provided method may elicit an additive anti-tumor effect.

In some embodiments, with respect to provided methods comprising administering to a subject an effective amount of a combination comprising a CD137/HER2 bispecific agent and a PD-1 axis inhibitor, the CD137/HER2 bispecific agent may comprise the amino acid sequences selected from the group consisting of SEQ ID NOs: 81 and 80, SEQ ID NOs: 79 and 82, SEQ ID NOs: 83 and 80, and SEQ ID NOs: 79 and 84, and/or the PD-1 axis inhibitor may comprise the amino acid sequences selected from the group consisting of SEQ ID NOs: 72 and 73, SEQ ID NOs: 74 and 75, and SEQ ID NOs: 76 and 77 or may be nivolumab, pembrolizumab, or atezolizumab.

In some embodiments, with respect to provided methods comprising administering to a subject an effective amount of a combination comprising a CD137/HER2 bispecific agent and a PD-1 axis inhibitor, the CD137/HER2 bispecific agent may comprise the amino acid sequences having at least 70%, at least 75%, at least 80%, at least 85%, at least 90%, at least 92%, at least 95%, at least 97%, at least 98%, at least 99%, or higher sequence identity to the amino acid sequences shown in SEQ ID NOs: 81 and 80, SEQ ID NOs: 79 and 82, SEQ ID NOs: 83 and 80, and SEQ ID NOs: 79 and 84, and/or the PD-1 axis inhibitor may comprise the amino acid sequences having at least 70%, at least 75%, at least 80%, at least 85%, at least 90%, at least 92%, at least 95%, at least 97%, at least 98%, at least 99%, or higher sequence identity to the amino acid sequences shown in SEQ ID NOs: 72 and 73, SEQ ID NOs: 74 and 75, and SEQ ID NOs: 76 and 77, or those of nivolumab, pembrolizumab, or atezolizumab. The meaning of sequence identities to bispecific fusion proteins comprising two amino acid chains as defined above applies mutatis mutandis to antibodies.

In some embodiments, with respect to provided methods comprising administering to a subject an effective amount of a combination comprising a CD137/HER2 bispecific agent and a PD-1 axis inhibitor, the combination is a combination from the group consisting of SEQ ID NOs: 81 and 80 and SEQ ID NOs: 72 and 73, SEQ ID NOs: 81 and 80 and SEQ ID NOs: 74 and 75, SEQ ID NOs: 81 and 80 and SEQ ID NOs: 76 and 77.

In some embodiments, provided methods result in a response in a subject. In some embodiments, a response is a partial response. In some embodiments, a response is a complete response. In some embodiments, a response is a sustained response (e.g., a sustained partial response or complete response) in the subject after cessation of a treatment.

In some embodiments, a subject of the disclosure may have been treated with a cancer therapy before the treatment of a combination comprising a CD137/HER2 bispecific agent and a PD-1 axis inhibitor. In some embodiments, a subject of the disclosure may have cancers that are resistant to one or more cancer therapies, where the cancers may be resistant at the beginning of treatment or it may become resistant during treatment. In some embodiments, a subject of the disclosure may have cancers that are resistant to trastuzumab. As described herein, the cancers may be at an early stage or at a late stage.

In some embodiments, a subject of the disclosure may suffer from advanced or metastatic breast cancer.

In some embodiments, provided methods include administration of an effective amount of an CD137/HER2 bispecific agent (e.g., SEQ ID NOs: 81 and 80, SEQ ID NOs: 79 and 82, SEQ ID NOs: 82 and 80, or SEQ ID NOs: 79 and 84). In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered to the individual at a dose of about 0.05 mg/kg to about 8 mg/kg bodyweight, about 0.15 mg/kg to about 8 mg/kg bodyweight, about 0.5 mg/kg to about 8 mg/kg bodyweight, about 1 mg/kg to about 8 mg/kg bodyweight, about 0.05 mg/kg to about 5 mg/kg bodyweight, about 0.15 mg/kg to about 5 mg/kg bodyweight, about 0.5 mg/kg to about 5 mg/kg bodyweight, or about 1 mg/kg to about 5 mg/kg bodyweight. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered to the individual at a dose of about 0.5 mg/kg to about 8 mg/kg bodyweight, about 1 mg/kg to about 8 mg/kg bodyweight, about 0.5 mg/kg to about 5 mg/kg bodyweight, or about 1 mg/kg to about 5 mg/kg bodyweight. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered to the individual at a dose of about 1 mg/kg to about 8 mg/kg bodyweight. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered to the individual at a dose of about 1 mg/kg to about 5 mg/kg bodyweight. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered at a dose of about 0.05 mg/kg bodyweight, about 0.15 mg/kg bodyweight, about 0.5 mg/kg bodyweight, about 1.0 mg/kg bodyweight, about 2.5 mg/kg bodyweight, about 5.0 mg/kg bodyweight, or about 8.0 mg/kg bodyweight. As a general proposition, the therapeutically effective amount of an CD137/HER2 bispecific agent (e.g., SEQ ID NOs: 81 and 80, SEQ ID NOs: 79 and 82, SEQ ID NOs: 82 and 80, or SEQ ID NOs: 79 and 84) administered to a human will be in the range of about 0.01 to about 50 mg/kg, whether by one or more administrations. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) may be administered from about weekly to every about 4 weeks, from about weekly to every about 3 weeks, or from about weekly to every about 2 weeks. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) may be administered about weekly, every about 2 weeks, every about 3 weeks, or every about 4 weeks. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) may be administered on days 1 of each cycle, on days 1 of each 7-day cycle, on days 1 and 8 of each 14-day cycle, on days 1, 8 and 15 of each 21-day cycle, or on days 1, 8, and 15 of each 28-day cycle. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) may be administered every about 3 weeks, on days 1 of each cycle. In some embodiments, at least one cycle, such as 2, 3, 4, 5, 10, 15, 20, 25, 30 or more cycles, are administered. In some embodiments, the provided CD137/HER2 bispecific agent has a sequence as set forth in SEQ ID NOs: 81 and 80.

In some embodiments, provided methods of the disclosure include administration of an effective amount of a PD-1 axis inhibitor selected from the group consisting of a PD-1 antagonist, a PD-L1 antagonist, and a PD-L2 antagonist.

In some embodiments, a PD-L1 antagonist is an antibody, such as an antibody that is capable of inhibiting PD-L1 binding to its binding partner(s), such as PD-1 and B7.1. In some embodiments, a PD-L1 antagonist is the PD-L1 antibody set forth in SEQ ID NOs: 76 and 77, which may be administered at a dose of about 800 mg to about 1500 mg from about weekly to every about 4 weeks, from about weekly to every about 3 weeks, from about weekly to every about 2 weeks, such as about weekly, every about 2 weeks, every about 3 weeks, or every about 4 weeks (e.g., about 1000 mg to about 1300 mg, e.g., about 1100 mg to about 1200 mg). In some embodiments, a PD-L1 antagonist is the PD-L1 antibody set forth in SEQ ID NOs: 76 and 77, which may be administered at a dose of about 800 mg to about 1500 mg every about three weeks (e.g., about 1000 mg to about 1300 mg every about three weeks, e.g., about 1100 mg to about 1200 mg every about three weeks). In some embodiments, the PD-L1 antibody set forth in SEQ ID NOs: 76 and 77 may be administered at a dose of about 1200 mg every about three weeks.

In some embodiments, a PD-1 antagonist is an antibody, such as an antibody that is capable of inhibiting PD-1 binding to its binding partner(s), such as PD-L1 and PD-L2. In some embodiments, a PD-1 antagonist is the PD-1 antibody set forth in SEQ ID NOs: 72 and 73 or SEQ ID NOs: 74 and 75, which may be administered at a dose of about 100 mg to about 600 mg every about one, two, there, four, five or six weeks (e.g., about 100 mg, 120 mg, 140 mg, 160 mg, 180 mg, 200 mg, 220 mg, 240 mg, 260 mg, 280 mg, 300 mg, 400 mg, 420 mg, 440 mg, 460 mg, 480 mg, 500 mg, 520 mg, 540 mg, 560 mg, or 600 1300 mg every about one, two, there, four, five or six weeks). In some embodiments, the PD-1 antibody set forth in SEQ ID NOs: 72 and 73 may be administered at a dose of about 240 mg every about two weeks. In some embodiments, the PD-1 antibody set forth in SEQ ID NOs: 72 and 73 may be administered at a dose of about 480 mg every about four weeks. In some embodiments, the PD-1 antibody set forth in SEQ ID NOs: 74 and 75 may be administered at a dose of about 200 mg every about three weeks.

As a general proposition, the therapeutically effective amount of a PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77) may be administered to a human will be in the range of about 0.01 to about 50 mg/kg of patient body weight whether by one or more administrations. In some embodiments, for example, a provided PD-1 axis inhibitor(e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77) may be administered in a dose of about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg. In some embodiments, a provided PD-1 axis inhibitor (e.g., a PD-L1 antagonist, such as the PD-L1 antibody set forth in SEQ ID NOs: 76 and 77) may be administered at about 15 mg/kg. In some embodiments, the PD-1 axis inhibitor set forth in SEQ ID NOs: 72 and 73 may be administered at about 3 mg/kg every about two weeks. In some embodiments, the PD-1 axis inhibitor set forth in SEQ ID NOs: 74 and 75 may be administered at about 2 mg/kg every about three weeks. Other dosage regimens may also be useful. In one embodiment, a provided PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77) may be administered to a human at a flat dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg, or about 1500 mg. In some embodiments, a provided PD-1 axis inhibitor (e.g., a PD-L1 antagonist, such as the PD-L1 antibody set forth in SEQ ID NOs: 76 and 77) may be administered at a dose of about 1150 mg to about 1250 mg every about three weeks. In some embodiments, a provided PD-1 axis inhibitor (e.g., a PD-L1 antagonist, such as the PD-L1 antibody set forth in SEQ ID NOs: 76 and 77) may be administered at a dose of about 1200 mg every about three weeks. In some embodiments, a provided PD-1 axis inhibitor (e.g., a PD-1 antagonist, such as the PD-1 antibody set forth in SEQ ID NOs: 72 and 73) may be administered at a dose of about 240 mg. In some embodiments, a provided PD-1 axis inhibitor (e.g., a PD-1 antagonist, such as the PD-1 antibody set forth in SEQ ID NOs: 74 and 75) may be administered at a dose of about 200 mg. In some embodiments, a dose of a provided PD-1 axis inhibitor of the disclosure may be administered as a single dose or as multiple doses (e.g., 2 or 3 doses). In some embodiments, a provided PD-1 axis inhibitor may be administered at a reduced dose, when administered in a combination treatment as compared to a single treatment. In some embodiments, for example, a provided method for treating or delaying progression of cancer in a subject comprises a dosing regimen comprising treatment cycles, wherein a subject is administered, on days 1 of each cycle, a human PD-1 axis binding antagonist (e.g., a PD-L1 antagonist, such as the PD-L1 antibody set forth in SEQ ID NOs: 76 and 77) at a dose of about 1200 mg, wherein each cycle is about 21 days (i.e., each cycle is repeated every about 21 days). In some embodiments, for example, a provided method for treating or delaying progression of cancer in a subject comprises a dosing regimen comprising treatment cycles, wherein a subject is administered, on days 1 of each cycle, a human PD-1 axis binding antagonist (e.g., a PD-1 antagonist, such as the PD-1 antibody set forth in SEQ ID NOs: 72 and 73) at a dose of about 240 mg wherein each cycle is about 14 days or at a dose about 480 mg wherein each cycle is about 28 days. In some embodiments, for example, a provided method for treating or delaying progression of cancer in a subject comprises a dosing regimen comprising treatment cycles, wherein a subject is administered, on days 1 of each cycle, a human PD-1 axis binding antagonist (e.g., a PD-1 antagonist, such as the PD-1 antibody set forth in SEQ ID NOs: 74 and 75) at a dose of about 240 mg, wherein each cycle is about 21 days. In some embodiments, at least one cycle, such as 2, 3, 4, 5, 10, 15, 20, 25, 30 or more cycles, are administered.

In some embodiments, a CD137/HER2 bispecific agent (e.g., SEQ ID NOs: 81 and 80, SEQ ID NOs: 79 and 82, SEQ ID NOs: 82 and 80, or SEQ ID NOs: 79 and 84) and a PD-1 axis inhibitor (e.g., a PD-L1 antagonist, such as the PD-L1 antibody set forth in SEQ ID NOs: 76 and 77) of the disclosure are administered in a single dosing regimen. The administration of these components may be sequentially (at different times) or concurrently (at the same time) within the context of the dosing regimen. For example, in some embodiments, methods of the disclosure comprises a dosing regimen comprising treatment cycles, wherein a subject is administered, on days 1 of each cycle, an CD137/HER2 bispecific agent (e.g., SEQ ID NOs: 81 and 80, SEQ ID NOs: 79 and 82, SEQ ID NOs: 82 and 80, or SEQ ID NOs: 79 and 84) at a dose of about 0.05 mg/kg bodyweight, 0.15 mg/kg bodyweight, 0.5 mg/kg bodyweight, 1.0 mg/kg bodyweight, 2.5 mg/kg bodyweight, 5.0 mg/kg bodyweight, or 8.0 mg/kg bodyweight, and immediately after, a PD-1 axis inhibitor (e.g., a PD-L1 antagonist, such as the PD-L1 antibody set forth in SEQ ID NOs: 76 and 77) is administered at a dose about 1200 mg. In some embodiments, each cycle is about 21 days. In some embodiments, at least one cycle, such as 2, 3, 4, 5, 10, 15, 20, 25, 30 or more cycles, are administered.

In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered in a separate composition as a provided PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77). In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) is administered in the same composition as a provided PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77).

In some embodiments, a CD137/HER2 bispecific agent (e.g., SEQ ID NOs: 81 and 80, SEQ ID NOs: 79 and 82, SEQ ID NOs: 82 and 80, or SEQ ID NOs: 79 and 84) and a PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77) of the disclosure are administered by the same route of administration. In some other embodiments, a CD137/HER2 bispecific agent (e.g., SEQ ID NOs: 81 and 80, SEQ ID NOs: 79 and 82, SEQ ID NOs: 82 and 80, or SEQ ID NOs: 79 and 84) and a PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77) of the disclosure are administered by the same route of administration or by different routes of administration. In some embodiments, a provided CD137/HER2 bispecific agent (e.g., SEQ ID NOs: 81 and 80, SEQ ID NOs: 79 and 82, SEQ ID NOs: 82 and 80, or SEQ ID NOs: 79 and 84) may be administered intravenously, intramuscularly, subcutaneously, topically, orally, transdermally, intraperitoneally, intraorbitally, by implantation, by inhalation, intrathecally, intraventricularly, or intranasally. In some embodiments, a provided PD-1 axis inhibitor (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77) may be administered intravenously, intramuscularly, subcutaneously, topically, orally, transdermally, intraperitoneally, intraorbitally, by implantation, by inhalation, intrathecally, intraventricularly, or intranasally.

In some embodiments, provided methods may further comprise an additional therapy. In some embodiments, an additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. Such additional therapy may be in the form of adjuvant or neoadjuvant therapy. In some embodiments, an additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.).

### E. Pharmaceutical Formulations

In some embodiments, molecules of the disclosure including provided combinations and components of combinations, such as CD137/HER2 bispecific agents (e.g., SEQ ID NOs: 81 and 80, SEQ ID NOs: 79 and 82, SEQ ID NOs: 82 and 80, or SEQ ID NOs: 79 and 84) and PD-1 axis inhibitors (e.g., a PD-1 antagonist, such as the PD-1 antibody set forth in SEQ ID NOs: 72 and 73 or SEQ ID NOs: 74 and 75, or a PD-L1 antagonist, such as the PD-L1 antibody set forth in SEQ ID NOs: 76 and 77), may be formulated in accordance with standard pharmaceutical practice for use as “active ingredients” of therapeutic compositions. Compositions comprising such molecules may contain one or more pharmaceutically acceptable carrier, glidant, diluent, or excipient, which facilitate administration of the composition and/or facilitate delivery of the composition to the site of action. Suitable carriers, diluents and excipients are known to those skilled in the art and include materials such as carbohydrates, waxes, water soluble and/or swellable polymers, hydrophilic or hydrophobic materials, gelatin, oils, solvents, water and the like. Compositions of the disclosure may be in any suitable form, for example tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders, to name just a few non-limiting alternatives. Such compositions (or formulations) may be prepared using methods known in the art, such as conventional dissolution and mixing procedures.

In some embodiments, formulations of the disclosure may be prepared for various routes and types of administration in the form of a lyophilized formulation, milled powder, or an aqueous solution.

In some embodiments, the CD137/HER2 bispecific agent is formulated as aqueous solution with a target protein concentration of about 25 mg/mL. In some embodiments, a commercial formulation of SEQ ID NOs: 76 and 77 may be used containing SEQ ID NOs: 76 and 77 at about 1200 mg/20 mL (60 mg/mL) in the form of a preservative-free solution for intravenous infusion.

Additional objects, advantages, and features of this disclosure will become apparent to those skilled in the art upon examination of the following Examples and the attached Figures thereof, which are not intended to be limiting. Thus, it should be understood that although the present disclosure is specifically disclosed by exemplary embodiments and optional features, modification and variation of the disclosures embodied therein herein disclosed may be resorted to by those skilled in the art and that such modifications and variations are considered to be within the scope of this disclosure.

### V. EXAMPLES

### Example 1

### Assessment of T-Cell Activation Induced by the Combination of a CD137/HER2 Bispecific Agent and a PD-1 Antibody

A T cell assay was employed to assess the ability of a CD137/HER2 bispecific agent shown in SEQ ID NOs: 81 and 80, when used in combination with an anti-PD-1 antibody at fixed molar ratio, to costimulate T-cell responses. For this purpose, the combination of the CD137/HER2 bispecific agent (SEQ ID NOs: 81 and 80) and a PD-1 antibody shown in SEQ ID NOs: 72 and 73 were added to staphylococcal enterotoxin B (SEB) stimulated human peripheral blood mononuclear cells (PBMCs), in the presence of the tumor cell line NCI-N87, and incubated for 3 days at 37° C. IL-2 secretion levels, as a demonstration of T-cell activation, were measured in the supernatants.

PBMCs from healthy volunteer donors were isolated from buffy coats by centrifugation through a polysucrose density gradient (Biocoll, 1.077 g/mL, Biochrom), following Biochrom's protocols. The purified PBMCs were resuspended in a buffer consisting of 90% FCS and 10% DMSO, immediately frozen down and stored in liquid nitrogen until further use. For the assay, PBMCs were thawed and rested in culture media (RPMI 1640, Life Technologies) supplemented with 10% FCS and 1% Penicillin-Streptomycin (Life Technologies) for 16 h at 37° C. in a humidified 5% CO₂ atmosphere.

The following procedure was performed using triplicates for each experimental condition: tumor cell line NCI-N87 were treated for 30 min at 37° C. with 30 μg/ml mitomycin C (Sigma Aldrich) in order to block proliferation. Mitomycin treated cells were then washed twice in culture medium and plated at 2.5×10⁴ cells per well onto a 384 well flat-bottom tissue culture plates to allow adhesion overnight at 37° C. in a humidified 5% CO₂ atmosphere. The target cells had before been grown under standard conditions, detached using Accutase (PAA Laboratories), and resuspended in culture media.

On the next days, after washing the plates twice with PBS, 2.5×10⁴ PBMCs per well were added to the tumor cells. A dilution series of a combination of the CD137/HER2 bispecific agent (SEQ ID NOs: 81 and 80) and the PD-1 antibody (SEQ ID NOs: 72 and 73) (1:10 molar ratio), the CD137/HER2 bispecific agent (SEQ ID NOs: 81 and 80) alone, or the PD-1 antibody (SEQ ID NOs: 72 and 73) alone, typically ranging from 0.001 nM to 100 nM, together with 0.05 ng/mL SEB, were added to the respective wells. Plates were covered with a gas permeable seal (4titude) and incubated at 37° C. in a humidified 5% CO₂ atmosphere for 3 days. Subsequently, IL-2 levels in the supernatant were assessed using the human IL-2 DuoSet kit (R&D Systems) as described in the following procedures.

384 well plates were coated for 2 h at room temperature with 1 μg/mL “Human IL-2 Capture Antibody” in PBS. Subsequently, wells were washed 5 times with 80 μl PBS supplemented with 0.05% Tween (PBS-T). After 1 h blocking in PBS-T containing 1% casein (w/w), assay supernatants and a concentration series of an IL-2 standard diluted in culture medium was transferred to respective wells and incubated overnight at 4° C. The next day, a mixture of 100 ng/mL goat anti-hlL-2-Bio detection antibody (R&D Systems) and 1 μg/mL Sulfotag-labelled streptavidin (Mesoscale Discovery) in PBS-T containing 0.5% casein were added and incubated at room temperature for 1 h. After washing, 25 μL reading buffer (Mesoscale Discovery) was added to each well and the resulting electrochemiluminescence (ECL) signal was detected using a Mesoscale Discovery reader. Analysis and quantification were performed using Mesoscale Discovery software.

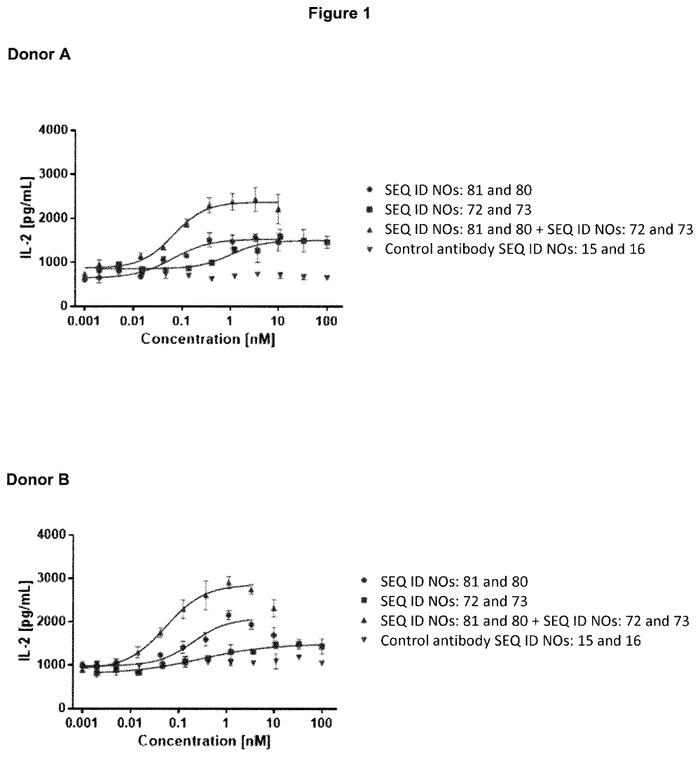

The result of representative experiments is depicted in FIG. 1 and the fitted EC₅₀s for induced IL-2 secretion is summarized in Table 1. An hIgG4 isotype control antibody was tested to set the basal activity. The combination of the CD137/HER2 bispecific agent (SEQ ID NOs: 81 and 80) and the PD-1 antibody (SEQ ID NOs: 72 and 73) were able to induce a dose-dependent secretion of IL-2, with an improved (lower) EC₅₀ values as compared to the CD137/HER2 bispecific agent or the PD-1 antibody alone. Meanwhile, IL-2 levels induced by the combination were higher compared to equimolar concentrations of the CD137/HER2 bispecific agent (SEQ ID NOs: 81 and 80) or the PD-1 antibody (SEQ ID NOs: 72 and 73) and displayed a synergistic or additive effect.

TABLE 1
Efficacy of inducing IL-2 secretion

| Molecule(s) | EC₅₀ (Donor A) | EC₅₀ (Donor B) |
|---|---|---|
| SEQ ID NOs: 81 and 80 | 0.1494 nM | 0.1165 nM |
| SEQ ID NOs: 72 and 73 | 0.4198 nM | 0.8538 nM |
| SEQ ID NOs: 81 and 80 + | 0.05462 nM | 0.05817 nM |
| SEQ ID NOs: 72 and 73 | | |

### Example 2

### Assessment of T-Cell Activation Induced by a CD137/HER2 Bispecific Agent in Combination with a PD-1 Antibody

Another T cell assay was employed to assess the ability of a CD137/HER2 bispecific agent shown in SEQ ID NOs: 81 and 80, when used in combination with a fixed concentration of a PD-1 antibody, to costimulate T-cell responses. For this purpose, the combination of a CD137/HER2 bispecific agent (SEQ ID NOs: 81 and 80) at various concentrations and a PD-1 antibody shown in SEQ ID NOs: 72 and 73 or in SEQ ID NOs: 74 and 75 at a fixed concentration were added to SEB stimulated human PBMCs, in the presence of the tumor cell line NCI-N87, and incubated for 3 days at 37° C. IL-2 secretion levels, as a demonstration of T-cell activation, were measured in the supernatants.

PBMCs from healthy volunteer donors were isolated and treated as described in Example 1. Tumor cell line NCI-N87 were treated and plated at 2.5×10⁴ cells per well the day before the assay as described in Example 1.

The following procedure was performed using triplicates for each experimental condition: NCI-N87 coated plates were washed twice with PBS, and 2.5×10⁴ PBMCs per well were added to the tumor cells. The CD137/HER2 bispecific agent (SEQ ID NOs: 81 and 80) at various concentrations, ranging from 0.0002 nM to 10 nM, alone or in combination with SEQ ID NOs: 72 and 73 or SEQ ID NOs: 74 and 75 at 10 nM or 100 nM were added to the respective wells, together with 1 ng/mL SEB. In the same experiment, the PD-1 antibody (SEQ ID NOs: 72 and 73 or SEQ ID NOs: 74 and 75) was also titrated alone for comparison. Plates were then covered with a gas permeable seal (4titude) and incubated at 37° C. in a humidified 5% CO₂ atmosphere for 3 days. Afterward, IL-2 levels in the supernatant were assessed as described in Example 1.

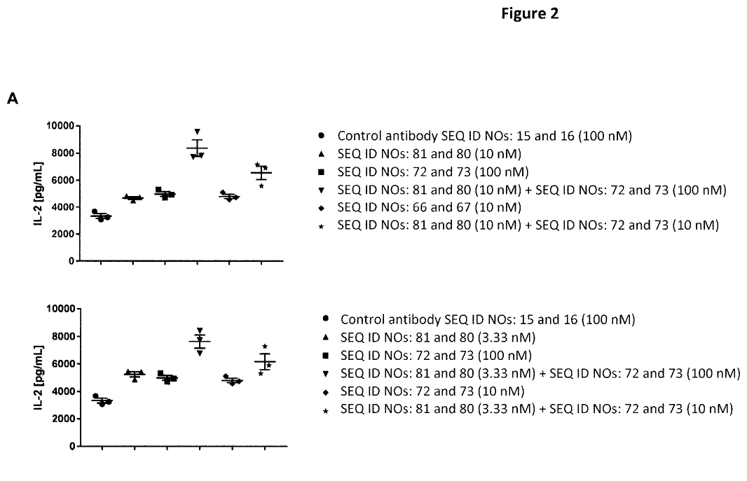

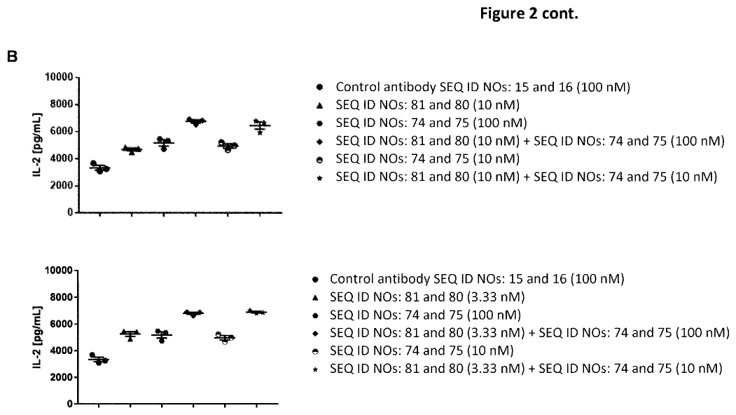

The results of representative experiments are depicted in FIG. 2 (the CD137/HER2 bispecific agent tested at 10 nM and 3.33 nM; data at other concentrations not shown). The combination of the CD137/HER2 bispecific agent (SEQ ID NOs: 81 and 80) with either of the tested PD-1 antibody (SEQ ID NOs: 72 and 73 and SEQ ID NOs: 74 and 75) induced higher IL-2 secretion and act synergistically or additively. When the CD137/HER2 bispecific agent (SEQ ID NOs: 81 and 80) was combined with SEQ ID NOs: 72 and 73, the induced IL-2 secretion levels were higher when 100 nM antibody was used than when 10 nM antibody was used. On the other hand, the concentration (at 100 nM or 10 nM) of SEQ ID NOs: 74 and 75 did not affect the IL-2 secretion when used in combination with the CD137/HER2 bispecific agent (SEQ ID NOs: 81 and 80).

### Example 3

### Assessment of T-Cell Activation Induced by a CD137/HER2 Bispecific Agent in Combination with a PD-L1 Antibody in the Presence of Tumor Cells Expressing Different Level of HER2 and/or PD-L1

To assess the ability of a combination of the CD137/HER2 bispecific agent shown in SEQ ID NOs: 81 and 80 and a PD-1 axis inhibitor to costimulate T-cell activation in a HER2 target dependent manner, a further T cell assay was employed. The combination of the CD137/HER2 bispecific agent (SEQ ID NOs: 81 and 80) different concentrations with a PD-L1 antibody shown in SEQ ID NOs: 76 and 77 at a fixed concentration was applied to SEB stimulated T cells, in the presence of a tumor cell line with different HER2 and/or PD-L1 expression levels. Tested tumor cell lines include NCI-N87 (HER2 high, PD-L1 low), JIMT-1 (HER2 moderate, PD-L1 moderate) and MDA-MB-231 (HER2 low, PD-L1 moderate).

PBMCs from healthy volunteer donors were isolated and treated as described in Example 1.

The following procedure was performed using triplicates for each experimental condition: the tumor cell lines NCI-N87, JIMT-1, and MDA-MB-231 were grown or treated with mitomycin C as described in Example 1 and plated at 8.3×10³ cells per well in culture medium to allow adhesion overnight at 37° C. in a humidified 5% CO₂ atmosphere. On the next days, after washing the plates coated with tumor cells twice with PBS, 2.5×10⁴ PBMCs per well were added to each well. The CD137/HER2 bispecific agent (SEQ ID NOs: 81 and 80) at various concentrations, ranging from 0.0002 nM to 10 nM, alone or in combination with the PD-L1 antibody (SEQ ID NOs: 76 and 77) or hIgG4 isotype control antibody at 100 nM were added to the respective wells, together with 0.05 ng/mL SEB. In the same experiment, the PD-L1 antibody also titrated alone for comparison. Plates were then covered with a gas permeable seal (4titude) and incubated at 37° C. in a humidified 5% CO₂ atmosphere for 3 days. Afterward, IL-2 levels in the supernatant were assessed as described in Example 1.

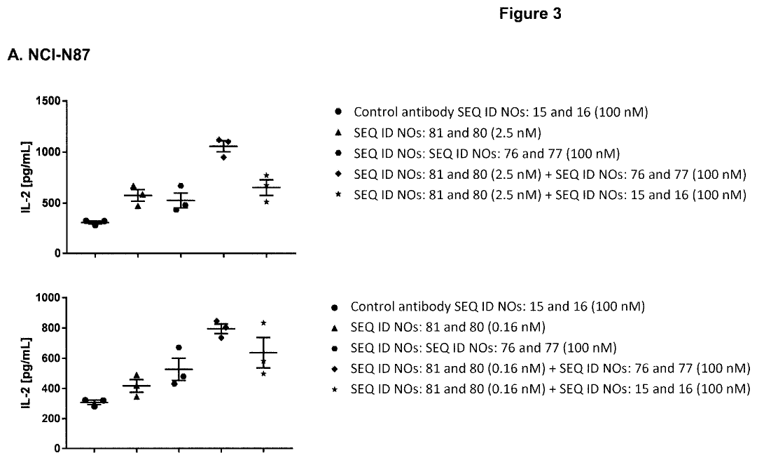

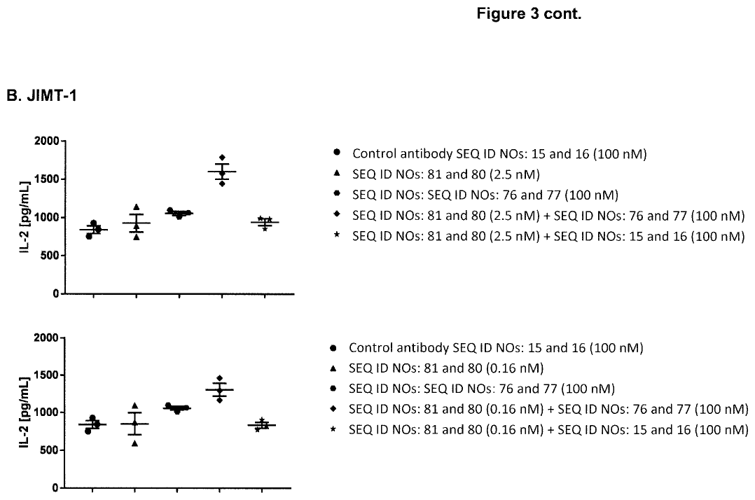

Exemplary data of the CD137/HER2 bispecific agent (SEQ ID NOs: 81 and 80) tested at the concentration of 2.5 nM and 0.16 nM are shown in FIG. 3 (data of the bispecific agent at other concentrations not shown). Co-culturing of T cells with NCI-N87 (HER2 high, PD-L1 low) or JIMT-1 (HER2 moderate, PD-L1 moderate) in presence of the combination of the CD137/HER2 bispecific agent (SEQ ID NOs: 81 and 80) and the PD-L1 antibody (SEQ ID NOs: 76 and 77) led to a statistically significant increased IL-2 secretion as compared to the bispecific agent or the antibody alone or a combination of the bispecific agent and an hIgG4 isotype control antibody (FIGS. 3A and 3B). Additionally, co-culturing with MDA-MB-231 (HER2 low, PD-L1 moderate) with the combination of the CD137/HER2 bispecific agent (SEQ ID NOs: 81 and 80) and the PD-L1 antibody (SEQ ID NOs: 76 and 77) induced higer IL-2 secretion, however, only to the same extend as when the antibody was used alone. This is likely resulting from the Her2 expression on MDA-MB-231 is not sufficient for the CD137/HER2 bispecific agent to have any effects.

Overall, the data indicate that the functional activity of the CD137/HER2 bispecific agent (SEQ ID NOs: 81 and 80) in combination with the PD-L1 antibody (SEQ ID NOs: 76 and 77), measured by their ability to activate T cells or increase IL-2 secretion, is synergistic or additive and depend on Her2 and PD-L1 expression profile on tumor cells.

### Example 4

### Assessment of Functional In Vivo Activity in a Xenograft Mouse Model Engrafted with Human PBMCs

In order to investigate the in vivo activity of a provided combination comprising a CD137/HER2 bispecific agent shown in SEQ ID NOs: 81 and 80 and a PD-1 axis inhibitor, cell line-derived xenograft mouse model is used. Accordingly, a human cancer cell line is implanted subcutaneously in immune deficient NOG mice, delivered at the age of 4-6 weeks with at least 1 week of quarantine. After the tumors have been reaching volumes of approximately 80-100 mm³, mice are substituted with human PBMCs. Test molecule(s) are injected at least three times and tumor growth and activity is constantly measured. After reaching study end, mice are sacrificed. Intratumoral infiltration of CD3-, CD4- and CD8-positive cells are assessed via immunohistochemistry. IFN-gamma RNAscope is conducted as further read-out.

### Example 5

### Assessment of Functional In Vivo Activity in a CD137 Humanized Mouse Model

In order to investigate the in vivo activity of a provided combination comprising a CD137/HER2 bispecific agent shown in SEQ ID NOs: 81 and 80 and a PD-1 axis inhibitor, a mouse cell line-derived xenograft mouse model over-expressing human Her2 is used. Accordingly, a mouse cancer cell line is implanted subcutaneously in humanized CD137 C57b mice, delivered at the age of 8-10 weeks with at least 1 week of quarantine. After the tumors have been reaching volumes of approximately 50-80 mm³, mice are randomized in homogeneous groups. Test molecule(s) are injected at least three times and tumor growth and activity will be constantly measured. After reaching study end, mice are sacrificed. Intratumoral infiltration of CD3-, CD4- and CD8-positive cells are assessed via immunohistochemistry. IFN-gamma RNAscope is conducted as further read-out.

### Example 6

### Clinical Study of CD137/HER2 Bispecific Agent in Combination with PD-1 Axis Inhibitor

Data discussed above indicate that a combination comprising a CD137/HER2 bispecific agent and a PD-1 axis inhibitor can act additively or synergistically to stimulate T cell activation in tumor microenvironment. Such a combination could therefore provide clinical benefit in patients with cancer.

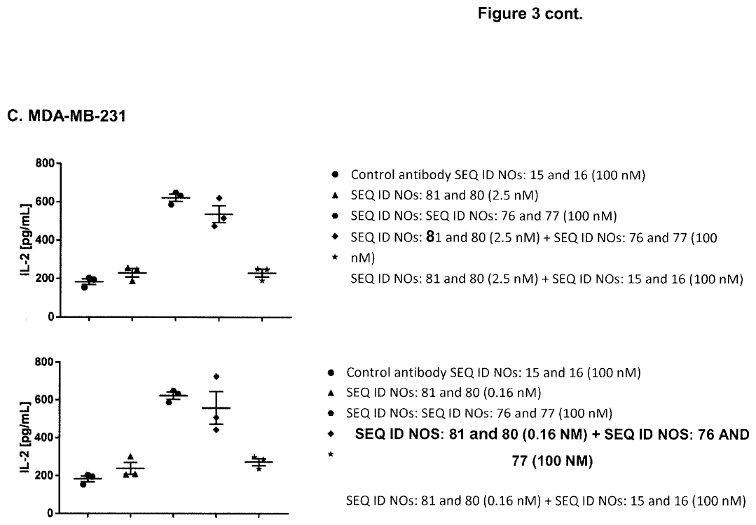

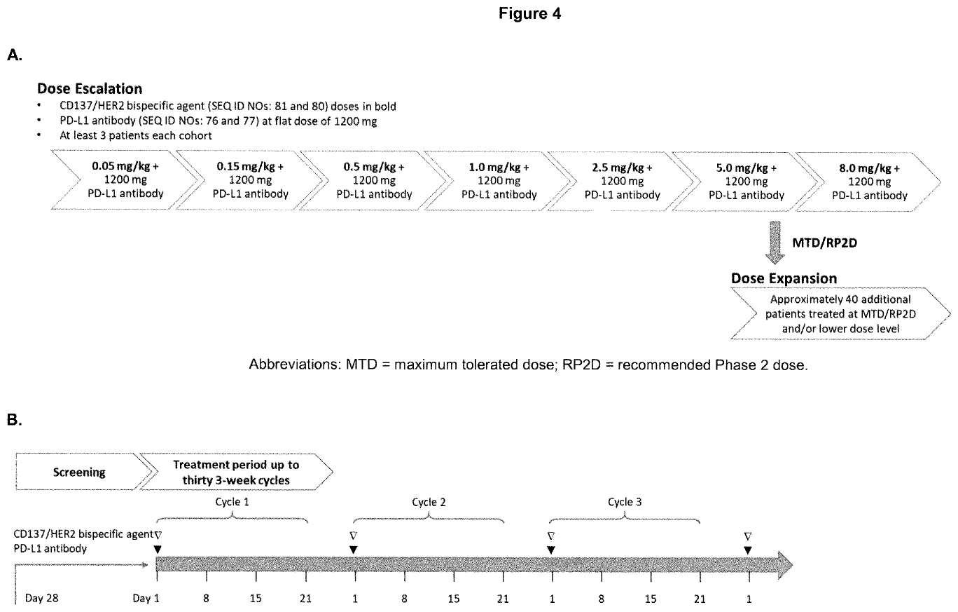

A Phase Ib, open-label, dose escalation study of a CD137/HER2 bispecific agent shown in SEQ ID NOs: 81 and 80 in combination with a PD-L1 antibody shown in SEQ ID NOs: 76 and 77 in patients with specific HER2-positive advanced or metastatic solid tumors is designed to determine the maximum tolerated dose (MTD) and Phase 2 dose (RP2D). Secondary objectives include assessing efficacy, safety, pharmacokinetics, and immunogenicity of the combination of the CD137/HER2 bispecific agent and the PD-L1 antibody. Exploratory objectives include assessing the preliminary antitumor activity of the combination, the CD137/HER2 bispecific agent exposure-response relationships, and biomarkers. The study includes a dose escalation period followed by an expansion period (FIG. 4A).

One treatment cycle is defined as 21 days and consists of intravenous infusions of the CD137/HER2 bispecific agent (SEQ ID NOs: 81 and 89) given every 3 weeks (Q3W) in combination with the PD-L1 antibody (SEQ ID NOs: 76 and 77) given Q3W (FIG. 4B). Patients receive the CD137/HER2 bispecific agent as a 2-hour intravenous infusion on Day 1 of each cycle, immediately followed by the PD-L1 antibody (1200 mg fixed dose) administered over 60 (±15) minutes. Subsequent doses of the PD-L1 antibody may be administered over 30 (±10) minutes if the first dose is well tolerated.

Dosing of the initial two patients at a given dose level is staggered by a minimum of 7 days. A safety review is conducted after the first patient has completed Day 8 visit. If no dose-limiting toxicities (DLTs) are observed for the first patient, staggering may be reduced to 72 hours for the second patient. No staggering is required for subsequent patients if no DLTs are observed in the first two patients. A DLT, as used herein, is defined as an adverse effect (AE) occurring in Cycle 1 for which other causes cannot be identified (and is therefore possibly related to study treatment) and satisfying at least one of the pre-set criteria. Toxicities are graded and documented according to the NCI CTCAE, version 4.03 guidelines. Patients who discontinue prior to completion of cycle 1 are replaced.

Dose escalation proceeds using a modified toxicity probability interval (mTPI) adaptive design to make dose recommendations and estimate the maximum tolerated dose (MTD) of the CD137/HER2 bispecific agent (SEQ ID NOs: 81 and 80) in combination with the approved dose of 1200 mg of the PD-L1 antibody (SEQ ID NOs: 76 and 77). The mTPI design is a model-based approach that has a pre-specified decision matrix that recommends escalating, reducing or maintaining the same dose or stopping dose escalation, based on the number of DLTs observed in the dose level under evaluation. The MTD is defined as the dose level that has an estimated probability of toxicity closest to 0.3. It is expected that 3 to 6 subjects will be enrolled in each dose escalation cohort. Intra-patient dose escalation is not permitted.

Dose escalation begins at a dose level of 0.05 mg/kg every 3 weeks (Q3W) in combination with a fixed dose of 1200 mg the PD-L1 antibody Q3W. After a minimum of 3 subjects within this dose have been treated at the target dose level and evaluated through the DLT evaluation period, if the mTPI model recommends escalation, then enrollment may begin for next dose level. The mTPI model's recommendations are based only on DLTs. However, all available safety data as well as emerging PK, persistence and pharmacodynamic data, and DLT modeling recommendations are considered in determining subsequent doses to be tested. Dose escalation continues until the MTD has been reached, until the maximum sample size (30 subjects) has been reached, or until a stopping rule for safety or underdosing takes effect, whichever occurs first. Dose levels of the CD137/HER2 bispecific agent and PD-L1 are tested during the dose escalation period per Table 2. The doses of the CD137/HER2 bispecific agent are adjusted or interrupted as appropriated based on treatment modifications.

TABLE 2
Doses of the CD137/HER2 bispecific agent and the PD-L1
antibody evaluated in the dose escalation period

| | | CD137/HER2 | PD-L1 antibody |
|---|---|---|---|
| | Minimum | bispecific agent | (SEQ ID |
| Dose | No. of | (SEQ ID NOs: 81 and 80) | NOs: 76 and 77) |
| Level | Patients | Dose (mg/kg) | Dose (mg) |
| 1 | 3 | 0.05 | 1200 |
| 2 | 3 | 0.15 | 1200 |
| 3 | 3 | 0.5 | 1200 |
| 4 | 3 | 1.0 | 1200 |
| 5 | 3 | 2.5 | 1200 |
| 6 | 3 | 5.0 | 1200 |
| 7 | 3 | 8.0 | 1200 |

Once the MTD has been established, the expansion period of the study investigates the preliminary efficacy and PD effects, in addition to safety and tolerability, of the RP2D established from the dose-escalation period.

Dosing continues until criteria for study treatment discontinuation are met (disease progression or withdrawal from the study) or up to 30 cycles. Patients are assessed for tumor response/progression every 6 weeks for the initial 24 weeks of dosing (first 8 cycles) and every 12 weeks after the Week 24 scans.

For the dose-finding cohorts, after the last patient in a given cohort has completed at least 1 cycle of study treatment, all safety data are reviewed to determine whether to continue or halt dose escalation, expand individual dose levels to gain additional safety data, determine the MTD and/or RP2D, or explore other dose levels/schedules.

Formulations—The CD137/HER2 bispecific agent (SEQ ID NOs: 81 and 80) is provided as aqueous solution in a 20-mL Type I USP/European Pharmacopeia glass vial with a nominal fill of 16 for infusion. The CD137/HER2 bispecific agent is formulated with a target protein concentration of 25 mg/mL in 20 mM Histidine, 250 mM Sorbitol, pH 6.3, 0.01% PS80. The PD-L1 antibody (SEQ ID NOs: 76 and 77) is provided as aqueous solution in a single-use, 20-cc Type 1 USP/European Pharmacopeia glass vial. The vial is designed to deliver 20 mL (1200 mg) of the PD-L1 antibody formulated as 60 mg/mL in 20 mM histidine acetate, 120 mM sucrose, 0.04% polysorbate 20, pH 5.8.

Safety assessment—The safety and tolerability of the CD137/HER2 bispecific agent and the PD-L1 antibody are assessed based on AEs (incidence and severity), performance status, physical examinations, 12-lead resting electrocardiograms (ECGs), LVEF assessments and laboratory safety evaluations. Laboratory abnormalities and AEs are graded according to NCI CTCAE v4.03. Management of immune-related (ir) AEs provides detailed guidance for the handling of immune-related (ir) AEs. AEs, ECGs, LVEF assessments and laboratory data are reviewed and summarized on an ongoing basis during the study. All patients who receive at least 1 dose of study treatment are included in the safety analyses.

Pharmacokinetic assessment—The single and multiple dose pharmacokinetic profiles of the CD137/HER2 bispecific agent and the PD-L1 antibody are investigated during the course of the study using the following parameters: (1) the integral of the concentration-time curve (area under the concentration-time curve, AUC); (2) maximum plasma, blood, serum, or other body fluid drug concentration (Cₘₐₓ); (3) time to reach Cmax (Tₘₐₓ); (4) terminal half-life (t_{1/2}); (5) the volume of plasma cleared of the drug per unit time clearance (clearance, CL); (6) the apparent volume of distribution during terminal phase (V_{z}); and (7) accumulation ratio (AR; AR=Cₘₐₓ (multiple dose)/Cₘₐₓ (single dose)).

For the assessment of the pharmacokinetics of the CD137/HER2 bispecific agent, peripheral venous blood (4 mL) is collected from all patients during Cycles 1 and 3 at pre-bispecific agent infusion, and at 5 minutes, 4, 8, 24, 48, 72, 168, and 336 hours after the end of bispecific agent infusion including flush. Samples are also collected pre-bispecific agent infusion on Day 1 of Cycles 2, 4-6, 8, 12, and 16 for quantitation of the bispecific agent.

For the assessment of the pharmacokinetics of the PD-L1 antibody, peripheral venous blood (4 mL) is collected from all patients during Cycle 1 at pre-antibody infusion, and at 30 minutes after the end of the PD-L1 antibody infusion including flush. Samples are also collected pre-antibody infusion on Day 1 of Cycles 2, 4, 8, 12, 16 for quantitation of the PD-L1 antibody.

Pharmacodynamic assessment—The pharmacodynamic response of the CD137/HER2 bispecific agent and the PD-L1 antibody are assessed by quantifying lymphocyte subtypes or markers in tumor biopsies or peripheral blood and cytokine levels in plasma. Selected pharmacodynamic markers planned for analysis (samples will be taken predose and postdose at pre-specified time points) are presented in Table 3.

TABLE 3
Pharmacodynamic markers evaluated

| Source | Marker |
|---|---|
| Biopsy | IHC (e.g. CD4, CD8, PDL-1, Ki67, FoxP3, etc.) |
| | Genomic analysis |
| Blood | Phenotyping (e.g. CD8 T cells, CD4 T cells) |
| Plasma | e.g., OD137, HER2, interleukin 8 (IL-8), IFN-gamma |

Biomarker assessment—Peripheral blood and fresh tumor tissue are collected prior to therapy and at selected time points on treatment (tumor biopsy). Residual sample material available after completion of the designated analyses are used for identification of additional pharmacodynamic or predictive markers or to enhance understanding of disease biology unless prohibited by local laws or regulations. Samples are de-identified to ensure patient privacy.

Efficacy assessment—The efficacy of the CD137/HER2 bispecific agent and the PD-L1 antibody is assessed by evaluating tumor response and progression according to RECIST, Version 1.1. Patients are assessed every 6 weeks for the initial 24 weeks of dosing (first 8 cycles). After the Week 24 scans, tumor assessments are conducted every 12 weeks. Tumor assessments would consist of clinical examination and appropriate imaging techniques, e.g., computerized tomography (CT) scans of the chest, abdomen, and pelvis with appropriate slice thickness per RECIST. Other studies such as magnetic resonance imaging (MRI), X-ray, positron emission tomography (PET) scan, and ultrasound are performed as required. The same methods used to detect lesions at baseline are used to follow the same lesions throughout the clinical study.

Immunogenicity assessment—The immunogenicity of the CD137/HER2 bispecific agent and the PD-L1 antibody is assessed based on antidrug antibody levels in the venous blood samples. Additionally, the concentration/adverse event—immunogenicity relationship was explored graphically, and tabulated to characterize a relationship between the changes from screening immunogenicity presence and serum concentration of the CD137/HER2 bispecific agent. The potential correlation between immunogenicity and other endpoints (major safety, efficacy and biomarker parameters) is also evaluated.

Statistical methods—Tabular summaries of data are descriptive in nature (i.e., number of patients [n], mean, standard deviation, median, minimum and maximum for continuous variables and n and percent for categorical variables). A more detailed description of analysis methods is provided in the statistical analysis plan (SAP) to be completed prior to the clinical database lock.

Embodiments illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present embodiments have been specifically disclosed by preferred embodiments and optional features, modification and variations thereof may be resorted to by those skilled in the art and that such modifications and variations are considered to be within the scope of this invention. All patents, patent applications, textbooks, and peer-reviewed publications described herein are hereby incorporated by reference in their entirety. Furthermore, where a definition or use of a term in a reference, which is incorporated by reference herein is inconsistent or contrary to the definition of that term provided herein, the definition of that term provided herein applies and the definition of that term in the reference does not apply. Each of the narrower species and subgeneric groupings falling within the generic disclosure also forms part of the invention. This includes the generic description of the invention with a proviso or negative limitation removing any subject matter from the genus, regardless of whether or not the excised material is specifically recited herein. In addition, where features are described in terms of Markush groups, those skilled in the art will recognize that the disclosure is also thereby described in terms of any individual member or subgroup of members of the Markush group. Further embodiments will become apparent from the following claims.

Equivalents: those skilled in the art will recognize, or be able to ascertain using no more than routine experimentation, many equivalents to the specific embodiments of the invention described herein. Such equivalents are intended to be encompassed by the following claims. All publications, patents, and patent applications mentioned in this specification are herein incorporated by reference into the specification to the same extent as if each individual publication, patent or patent application was specifically and individually indicated to be incorporated herein by reference.

### NON-PATENT REFERENCES

1. VINAY, D. S. & KWON, B. S. 2012. Immunotherapy of cancer with 4-1BB. Mol Cancer Ther, 11, 1062-70.
2. BARTKOWIAK, T. & CURRAN, M. A. 2015. 4-1BB Agonists: Multi-Potent Potentiators of Tumor Immunity. Front Oncol, 5, 117.
3. WILLIAMS, J. B., HORTON, B. L., ZHENG, Y., DUAN, Y., POWELL, J. D. & GAJEWSKI, T. F. 2017. The EGR2 targets LAG-3 and 4-1BB describe and regulate dysfunctional antigen-specific CD8+T cells in the tumor microenvironment. J Exp Med, 214, 381-400.
4. WILCOX, R. A., TAMADA, K., FLIES, D. B., ZHU, G., CHAPOVAL, A. I., BLAZAR, B. R., KAST, W. M. & CHEN, L. 2004. Ligation of CD137 receptor prevents and reverses established anergy of CD8+ cytolytic T lymphocytes in vivo. Blood, 103, 177-84.
5. TOLCHER, A. W., SZNOL, M., HU-LIESKOVAN, S., PAPADOPOULOS, K. P., PATNAIK, A., RASCO, D. W., DI GRAVIO, D., HUANG, B., GAMBHIRE, D., CHEN, Y., THALL, A. D., PATHAN, N., SCHMIDT, E. V. & CHOW, L. Q. M. 2017. Phase 1b Study of Utomilumab (PF-05082566), a 4-1BB/CD137 Agonist, in Combination with Pembrolizumab (MK-3475) in Patients with Advanced Solid Tumors. Clin Cancer Res, 23, 5349-5357.
6. SEGAL, N. H., HE, A. R., DOI, T., LEVY, R., BHATIA, S., PISHVAIAN, M. J., CESARI, R., CHEN, Y., DAVIS, C. B., HUANG, B., THALL, A. D. & GOPAL, A. K. 2018. Phase I Study of Single-Agent Utomilumab (PF-05082566), a 4-1BB/CD137 Agonist, in Patients with Advanced Cancer. Clin Cancer Res, 24, 1816-1823.
7. SZNOL, M., HODI, F. S., MARGOLIN, K., MCDERMOTT, D. F., ERNSTOFF, M. S., KIRKWOOD, J. M., WOJTASZEK, C., FELTQUATE, D. & LOGAN, T. 2008. Phase I study of BMS-663513, a fully human anti-CD137 agonist monoclonal antibody, in patients (pts) with advanced cancer (CA). Journal of Clinical Oncology, 26, 3007-3007.
8. MAKKOUK, A., CHESTER, C. & KOHRT, H. E. 2016. Rationale for anti-CD137 cancer immunotherapy. Eur J Cancer, 54, 112-119.
9. ALIZADEH, A. A., GENTLES, A. J., ALENCAR, A. J., LIU, C. L., KOHRT, H. E., HOUOT, R., GOLDSTEIN, M. J., ZHAO, S., NATKUNAM, Y., ADVANI, R. H., GASCOYNE, R. D., BRIONES, J., TIBSHIRANI, R. J., MYKLEBUST, J. H., PLEVRITIS, S. K., LOSSOS, I. S. & LEVY, R. 2011. Prediction of survival in diffuse large B-cell lymphoma based on the expression of 2 genes reflecting tumor and microenvironment. Blood, 118, 1350-8.
10. SHARPE, A. H., WHERRY, E. J., AHMED, R. & FREEMAN, G. J. 2007. The function of programmed cell death 1 and its ligands in regulating autoimmunity and infection. Nat Immunol, 8, 239-45.
11. GREENWALD, R. J., FREEMAN, G. J. & SHARPE, A. H. 2005. The B7 family revisited. Annu Rev Immunol, 23, 515-48.
12. GALON, J., COSTES, A., SANCHEZ-CABO, F., KIRILOVSKY, A., MLECNIK, B., LAGORCE-PAGES, C., TOSOLINI, M., CAMUS, M., BERGER, A., WIND, P., ZINZINDOHOUE, F., BRUNEVAL, P., CUGNENC, P. H., TRAJANOSKI, Z., FRIDMAN, W. H. & PAGES, F. 2006. Type, density, and location of immune cells within human colorectal tumors predict clinical outcome. Science, 313, 1960-4.
13. DONG, H., STROME, S. E., SALOMAO, D. R., TAMURA, H., HIRANO, F., FLIES, D. B., ROCHE, P. C., LU, J., ZHU, G., TAMADA, K., LENNON, V. A., CELIS, E. & CHEN, L. 2002. Tumor-associated B7-H1 promotes T-cell apoptosis: a potential mechanism of immune evasion. Nat Med, 8, 793-800.
14. BLANK, C., BROWN, I., PETERSON, A. C., SPIOTTO, M., IWAI, Y., HONJO, T. & GAJEWSKI, T. F. 2004. PD-L1/B7H-1 inhibits the effector phase of tumor rejection by T cell receptor (TCR) transgenic CD8+T cells. Cancer Res, 64, 1140-5.
15. IWAI, Y., TERAWAKI, S. & HONJO, T. 2005. PD-1 blockade inhibits hematogenous spread of poorly immunogenic tumor cells by enhanced recruitment of effector T cells. Int Immunol, 17, 133-44.
16. HIRANO, F., KANEKO, K., TAMURA, H., DONG, H., WANG, S., ICHIKAWA, M., RIETZ, C., FLIES, D. B., LAU, J. S., ZHU, G., TAMADA, K. & CHEN, L. 2005. Blockade of B7-H1 and PD-1 by monoclonal antibodies potentiates cancer therapeutic immunity. Cancer Res, 65, 1089-96.
17. HAMANISHI, J., MANDAI, M., IWASAKI, M., OKAZAKI, T., TANAKA, Y., YAMAGUCHI, K., HIGUCHI, T., YAGI, H., TAKAKURA, K., MINATO, N., HONJO, T. & FUJII, S. 2007. Programmed cell death 1 ligand 1 and tumor-infiltrating CD8+ T lymphocytes are prognostic factors of human ovarian cancer. Proc Natl Acad Sci USA, 104, 3360-5.
18. TOPALIAN, S. L., DRAKE, C. G. & PARDOLL, D. M. 2015. Immune checkpoint blockade: a common denominator approach to cancer therapy. Cancer Cell, 27, 450-61.
19. BU, X., MAHONEY, K. M. & FREEMAN, G. J. 2016. Learning from PD-1 Resistance: New Combination Strategies. Trends Mol Med, 22, 448-451.
20. BAI, J., GAO, Z., LI, X., DONG, L., HAN, W. & NIE, J. 2017. Regulation of PD-1/PD-L1 pathway and resistance to PD-1/PD-L1 blockade. Oncotarget, 8, 110693-110707.
21. SHOUSHTARI, A. N., FRIEDMAN, C. F., NAVID-AZARBAIJANI, P., POSTOW, M. A., CALLAHAN, M. K., MOMTAZ, P., PANAGEAS, K. S., WOLCHOK, J. D. & CHAPMAN, P. B. 2018. Measuring Toxic Effects and Time to Treatment Failure for Nivolumab Plus Ipilimumab in Melanoma. JAMA Oncol, 4, 98-101.
22. HAANEN, J., CARBONNEL, F., ROBERT, C., KERR, K. M., PETERS, S., LARKIN, J., JORDAN, K. & COMMITTEE, E. G. 2017. Management of toxicities from immunotherapy: ESMO Clinical Practice Guidelines for diagnosis, treatment and follow-up. Ann Oncol, 28, iv119-iv142.
23. SLAMON, D. J., LEYLAND-JONES, B., SHAK, S., FUCHS, H., PATON, V., BAJAMONDE, A., FLEMING, T., EIERMANN, W., WOLTER, J., PEGRAM, M., BASELGA, J. & NORTON, L. 2001. Use of chemotherapy plus a monoclonal antibody against HER2 for metastatic breast cancer that overexpresses HER2. N Engl J Med, 344, 783-92.
24. WARD, E. S., GUSSOW, D., GRIFFITHS, A. D., JONES, P. T. & WINTER, G. 1989. Binding activities of a repertoire of single immunoglobulin variable domains secreted from Escherichia coli. Nature, 341, 544-6.
25. HOLLIGER, P., PROSPERO, T. & WINTER, G. 1993. “Dia bodies”: small bivalent and bispecific antibody fragments. Proc Natl Acad Sci USA, 90, 6444-8.
26. JOHNSON, G. & WU, T. T. 2000. Kabat database and its applications: 30 years after the first variability plot. Nucleic Acids Res, 28, 214-8.
27. EHRENMANN, F., KAAS, Q. & LEFRANC, M. P. 2010. IMGT/3Dstructure-DB and IMGT/DomainGapAlign: a database and a tool for immunoglobulins or antibodies, T cell receptors, MHC, IgSF and MhcSF. Nucleic Acids Res, 38, D301-7.
28. SKERRA, A. 2000. Lipocalins as a scaffold. Biochim Biophys Acta, 1482, 337-50.
29. FLOWER, D. R., NORTH, A. C. & SANSOM, C. E. 2000. The lipocalin protein family: structural and sequence overview. Biochim Biophys Acta, 1482, 9-24.
30. FLOWER, D. R. 1996. The lipocalin protein family: structure and function. Biochem J, 318 (Pt 1), 1-14.
31. YAO, S., ZHU, Y. & CHEN, L. 2013. Advances in targeting cell surface signalling molecules for immune modulation. Nat Rev Drug Discov, 12, 130-46.
32. SNELL, L. M., LIN, G. H., MCPHERSON, A. J., MORAES, T. J. & WATTS, T. H. 2011. T-cell intrinsic effects of GITR and 4-1BB during viral infection and cancer immunotherapy. Immunol Rev, 244, 197-217.
33. BULLIARD, Y., JOLICOEUR, R., ZHANG, J., DRANOFF, G., WILSON, N. S. & BROGDON, J. L. 2014. OX40 engagement depletes intratumoral Tregs via activating FcgammaRs, leading to antitumor efficacy. Immunol Cell Biol, 92, 475-80.
34. BULLIARD, Y., JOLICOEUR, R., WINDMAN, M., RUE, S. M., ETTENBERG, S., KNEE, D. A., WILSON, N. S., DRANOFF, G. & BROGDON, J. L. 2013. Activating Fc gamma receptors contribute to the antitumor activities of immunoregulatory receptor-targeting antibodies. J Exp Med, 210, 1685-93.
35. KARACHALIOU, N., GONZALEZ-CAO, M., SOSA, A., BERENGUER, J., BRACHT, J. W. P., ITO, M. & ROSELL, R. 2017. The combination of checkpoint immunotherapy and targeted therapy in cancer. Ann Transl Med, 5, 388.
36. OTT, P. A., HODI, F. S., KAUFMAN, H. L., WIGGINTON, J. M. & WOLCHOK, J. D. 2017. Combination immunotherapy: a road map. J Immunother Cancer, 5, 16.
37. DAI, M., YIP, Y. Y., HELLSTROM, I. & HELLSTROM, K. E. 2015. Curing mice with large tumors by locally delivering combinations of immunomodulatory antibodies. Clin Cancer Res, 21, 1127-38.
38. WEI, H., ZHAO, L., HELLSTROM, I., HELLSTROM, K. E. & GUO, Y. 2014. Dual targeting of CD137 co-stimulatory and PD-1 co-inhibitory molecules for ovarian cancer immunotherapy. Oncoimmuno/ogy, 3, e28248.
39. CHEN, S., LEE, L. F., FISHER, T. S., JESSEN, B., ELLIOTT, M., EVERING, W., LOGRONIO, K., TU, G. H., TSAPARIKOS, K., LI, X., WANG, H., YING, C., XIONG, M., VANARSDALE, T. & LIN, J. C. 2015. Combination of 4-1BB agonist and PD-1 antagonist promotes antitumor effector/memory CD8 T cells in a poorly immunogenic tumor model. Cancer Immunol Res, 3, 149-60.
40. KOHRT, H. E., COLEVAS, A. D., HOUOT, R., WEISKOPF, K., GOLDSTEIN, M. J., LUND, P., MUELLER, A., SAGIV-BARFI, I., MARABELLE, A., LIRA, R., TROUTNER, E., RICHARDS, L., RAJAPASKA, A., HEBB, J., CHESTER, C., WALLER, E., OSTASHKO, A., WENG, W. K., CHEN, L., CZERWINSKI, D., FU, Y. X., SUNWOO, J. & LEVY, R. 2014. Targeting CD137 enhances the efficacy of cetuximab. J Clin Invest, 124, 2668-82.
41. KOHRT, H. E., HOUOT, R., WEISKOPF, K., GOLDSTEIN, M. J., SCHEEREN, F., CZERWINSKI, D., COLEVAS, A. D., WENG, W. K., CLARKE, M. F., CARLSON, R. W., STOCKDALE, F. E., MOLLICK, J. A., CHEN, L. & LEVY, R. 2012. Stimulation of natural killer cells with a CD137-specific antibody enhances trastuzumab efficacy in xenotransplant models of breast cancer. J Clin Invest, 122, 1066-75.
42. MORALES-KASTRESANA, A., CATALAN, E., HERVAS-STUBBS, S., PALAZON, A., AZPILIKUETA, A., BOLANOS, E., ANEL, A., PARDO, J. & MELERO, I. 2013. Essential complicity of perforin-granzyme and FAS-L mechanisms to achieve tumor rejection following treatment with anti-CD137 mAb. J immunother Cancer, 1, 3.
43. MASSARELLI, E. Clinical safety and efficacy assessment of the CD137 agonist urelumab alone and in combination with nivolumab in patients with hematologic and solid tumor malignancies. 31st Annual Meeting and Associated Programs of the Society for Immunotherapy of Cancer, 2016 National Harbor, Md.
44. SRIVASTAVA, R. M., TRIVEDI, S., CONCHA-BENAVENTE, F., GIBSON, S. P., REEDER, C., FERRONE, S. & FERRIS, R. L. 2017. CD137 Stimulation Enhances Cetuximab-Induced Natural Killer: Dendritic Cell Priming of Antitumor T-Cell Immunity in Patients with Head and Neck Cancer. Clin Cancer Res, 23, 707-716.
45. SILVA, J. P., VETTERLEIN, O., JOSE, J., PETERS, S. & KIRBY, H. 2015. The S228P mutation prevents in vivo and in vitro IgG4 Fab-arm exchange as demonstrated using a combination of novel quantitative immunoassays and physiological matrix preparation. J Biol Chem, 290, 5462-9.
46. GLAESNER, W., VICK, A. M., MILLICAN, R., ELLIS, B., TSCHANG, S. H., TIAN, Y., BOKVIST, K., BRENNER, M., KOESTER, A., PORKSEN, N., ETGEN, G. & BUMOL, T. 2010. Engineering and characterization of the long-acting glucagon-like peptide-1 analogue LY2189265, an Fc fusion protein. Diabetes Metab Res Rev, 26, 287-96.
47. DALL'ACQUA, W. F., KIENER, P. A. & WU, H. 2006. Properties of human IgG1s engineered for enhanced binding to the neonatal Fc receptor (FcRn). J Biol Chem, 281, 23514-24.
48. ZALEVSKY, J., CHAMBERLAIN, A. K., HORTON, H. M., KARKI, S., LEUNG, I. W., SPROULE, T. J., LAZAR, G. A., ROOPENIAN, D. C. & DESJARLAIS, J. R. 2010. Enhanced antibody half-life improves in vivo activity. Nat Biotechnol, 28, 157-9.

1. A method of treating cancer in a subject, comprising administering to the subject:
(a) a CD137/HER2 bispecific agent comprising (i) CDR1, CDR2, CDR3 domains of the heavy chain variable region having the sequence set forth in SEQ ID NO: 64, (ii) CDR1, CDR2, CDR3 domains of the light chain variable region having the sequence set forth in SEQ ID NO: 65, and (iii) a lipocalin mutein with the sequence set forth in any one of SEQ ID NOs: 21-39, and
(b) a PD-1 axis inhibitor.

2. A method of treating cancer in a subject, comprising:
(a) administering to the subject a CD137/HER2 bispecific agent, wherein the subject is also receiving a PD-1 axis inhibitor, so that the subject receives therapy in both, or
(b) administering to the subject a PD-1 axis inhibitor, wherein the subject is also receiving a CD137/HER2 bispecific agent, so that the subject receives therapy in both,
wherein the CD137/HER2 bispecific agent comprises (i) CDR1, CDR2, CDR3 domains of the heavy chain variable region having the sequence set forth in SEQ ID NO: 64, (ii) CDR1, CDR2, CDR3 domains of the light chain variable region having the sequence set forth in SEQ ID NO: 65, and (iii) a lipocalin mutein with the sequence set forth in any one of SEQ ID NOs: 21-39.

3. The method of claim 1 or 2, wherein the cancer is HER-2 positive and/or PD-L1 positive.

4. The method of any one of claims 1-3, wherein the method is capable of providing an enhanced anti-tumor effect.

5. The method of any one of claims 1-4, wherein the method is capable of providing an additive an-tumor effect as compared to the CD137/HER2 bispecific agent or the PD-1 axis inhibitor alone.

6. The method of any one of claims 1-4, wherein the method is capable of providing an synergistic an-tumor effect as compared to the CD137/HER2 bispecific agent or the PD-1 axis inhibitor alone.

7. The method of any one of claims 4-6, wherein the anti-tumor effect is selected from the group consisting of:
(a) stimulation of tumor-related immune response;
(b) increased IL-2 secretion;
(c) increased IL-2 secretion in a tumor microenvironment;
(d) increased IFN-gamma secretion;
(e) increased IFN-gamma secretion in a tumor microenvironment;
(f) expansion of CD4⁺ T cells;
(g) expansion of CD4⁺ T cells in a tumor microenvironment;
(h) expansion of CD8⁺ T cells;
(i) expansion of CD8⁺ T cells in a tumor microenvironment;
(j) expansion of tumor-infiltrating lymphocytes;
(k) activation of NK cells and increased antibody-dependent cell-mediated cytotoxicity (ADCC);
(l) activation of NK cells and increased ADCC in a tumor microenvironment;
(m) increased level of CD4 in a tumor microenvironment;
(n) decreased level of CD4 in a tumor microenvironment;
(o) increased level of CD8 in a tumor microenvironment;
(p) decreased level of CD8 in a tumor microenvironment;
(q) increased level of PD-L1 in a tumor microenvironment;
(r) decreased level of PD-L1 in a tumor microenvironment;
(s) increased level of Ki67 in a tumor microenvironment;
(t) decreased level of Ki67 in a tumor microenvironment;
(u) increased level of CD137 in a tumor microenvironment;
(v) decreased level of CD137 in a tumor microenvironment;
(w) increased level of HER2 in a tumor microenvironment;
(x) decreased level of HER2 in a tumor microenvironment;
(y) increased level of IL-8 in a tumor microenvironment;
(z) decreased level of IL-8 in a tumor microenvironment;
(aa) increased level of FoxP3 in a tumor microenvironment;
(ab) decreased level of FoxP3 in a tumor microenvironment;
(ac) reduction of tumor size;
(ad) suppression of tumor growth;
(ae) suppression of tumor metastasis;
(af) delay of recurrence; or
(ag) improved overall survival.

8. The method of any one of claims 1-7, wherein the method comprises at least one administration cycle, wherein the cycle is a period of about three weeks, wherein for each of the at least one cycles at least one dose of the CD137/HER2 bispecific agent is administered and at least one dose of the PD-1 axis inhibitor is administered.

9. The method of any one of claims 1-8, wherein the CD137/HER2 bispecific agent and the PD-1 axis inhibitor are administered sequentially or concurrently.

10. The method of any one of claims 1-9, wherein the CD137/HER2 bispecific agent is administered at a dose of about 0.05 mg/kg, 0.15 mg/kg, 0.5 mg/kg, 1.0 mg/kg, 2.5 mg/kg, 5.0 mg/kg, or 8.0 mg/kg.

11. The method of any one of claims 1-10, wherein the PD-1 axis inhibitor is an anti-PD-L1 antibody.

12. The method of claim 11, wherein the anti-PD-L1 antibody has the sequences set forth in SEQ ID NOs: 76 and 77.

13. The method of claim 11 or 12, wherein the anti-PD-L1 antibody is administered at a dose of about 1200 mg.

14. The method of any one of claims 1-10, wherein the PD-1 axis inhibitor is an anti-PD-1 antibody.

15. The method of any one of claims 1-10, wherein the PD-1 axis inhibitor is an antibody having the sequences set forth in SEQ ID NOs: 72 and 73 or SEQ ID NOs: 74 and 75.

16. The method of any one of claims 1-10, wherein the PD-1 axis inhibitor is atezolizumab, durvalulmab, avelumab, nivolumab, pembrolizumab, tislelizumab, or cemiplimab.

17. The method of any one of claims 1-16, wherein the CD137/HER2 bispecific agent and the PD-1 axis inhibitor are administered at the following doses:
(a) about 0.05 mg/kg CD137/HER2 bispecific agent and about 1200 mg PD-1 axis inhibitor,
(b) about 0.15 mg/kg CD137/HR2 bispecific agent and about 1200 mg PD-1 axis inhibitor,
(c) about 0.5 mg/kg CD137/HER2 bispecific agent and about 1200 mg PD-1 axis inhibitor,
(d) about 1.0 mg/kg CD137/HER2 bispecific agent and about 1200 mg PD-1 axis inhibitor,
(e) about 2.5 mg/kg CD137/HER2 bispecific agent and about 1200 mg PD-1 axis inhibitor,
(f) about 5.0 mg/kg CD137/HER2 bispecific agent and about 1200 mg PD-1 axis inhibitor, or
(g) about 8.0 mg/kg CD137/HER2 bispecific agent and about 1200 mg PD-1 axis inhibitor

18. The method of any one of claims 1-17, wherein the method comprises at least one administration cycle, such as 2, 3, 4, 5, 10, 15, 20, 25, and 30 cycles, wherein the cycle is a period of about three weeks.

19. The method of any one of claims 1-18, wherein the method comprises administering one dose of the CD137/HER2 bispecific agent and one dose of the PD-1 axis inhibitor, sequentially with respect to each other, on days 1 of each three-week cycle.

20. The method of any one of claims 1-19, wherein the method comprises administering one dose of the CD137/HER2 bispecific agent and one dose of the PD-1 axis inhibitor, sequentially with respect to each other, on days 1 of each cycle for 30 three-week cycles.

21. The method of any one of claims 1-20, wherein the CD137/HER2 bispecific agent comprises a lipocalin mutein specific for CD137 fused at the N-terminus via a linker to the C-terminus of each heavy chain of anti-HER2 antibody.

22. The method of any one of claims 21, wherein the lipocalin mutein specific for CD137 has the sequence set forth in SEQ ID NO: 22.

23. The method of any one of claims 22, wherein the anti-HER2 antibody has the sequence set forth in SEQ ID NOs: 79 and 80.

24. The method of any one of claims 1-23, wherein the CD137/HER2 bispecific agent has the sequences set forth in SEQ ID NOs: 81 and 80, SEQ ID NOs: 79 and 82, SEQ ID NOs:
83 and 80, or SEQ ID NOs: 79 and 84.

25. The method of any one of claims 1-24, wherein the cancer is HER-2 positive advanced or metastatic solid tumors.

26. The method of any one of claims 1-25, wherein the subject is previously treated.

27. The method of any one of claims 1-26, wherein the subject is previously treated with an anti-HER2 therapy.

28. The method of any one of claims 1-27, wherein the subject is previously treated with a PD-1 axis inhibitor therapy.

29. The method of any one of claims 1-28, wherein the treatment produces at least one effect chosen from stimulation of tumor-specific immune responses, reduction in tumor size, suppression of the growth of tumor cells, suppression of the metastasis, complete remission, partial remission, stabilization of disease, extension of the term before recurrence, extension of survival time, complete response, and partial response.

30. A combination comprising an CD137/HER2 bispecific agent antibody comprising (i) CDR1, CDR2, CDR3 domains of the heavy chain variable region having the sequence set forth in SEQ ID NO: 64, (ii) CDR1, CDR2, CDR3 domains of the light chain variable region having the sequence set forth in SEQ ID NO: 65, and (iii) a lipocalin mutein with the sequence set forth in any one of SEQ ID NOs: 21-39, and a PD-1 axis inhibitor, wherein the combination is suitable to be administered to a subject in at least one cycle, wherein for each cycle the CD137/HER2 bispecific agent is administered at a dose of about 0.05 mg/kg, 0.15 mg/kg, 0.5 mg/kg, 1.0 mg/kg, 2.5 mg/kg, 5.0 mg/kg, or 8.0 mg/kg and the anti-PD-L1 axis inhibitor is administered at a dose of about 1200 mg.

31. The combination of claim 30, wherein the CD137/HER2 bispecific agent and the anti-PD-L1 antibody are administered sequentially or concurrently.

32. The combination of claim 30 or 31, wherein the combination may be used for treating HER2-positive and/or PD-L1 positive tumors.

33. The combination of any one of claims 30-32, wherein the combination may produce enhanced anti-tumor effects.

34. The combination of any one of claims 30-33, wherein the combination may produce additive anti-tumor effects as compared to the CD137/HER2 bispecific agent or the PD-1 axis inhibitor alone.

35. The combination of any one of claims 30-33, wherein the combination may produce synergistic anti-tumor effects as compared to the CD137/HER2 bispecific agent or the PD-1 axis inhibitor alone.

36. A kit of parts comprising
(a) a pharmaceutical composition comprising an CD137/HER2 bispecific agent antibody comprising (i) CDR1, CDR2, CDR3 domains of the heavy chain variable region having the sequence set forth in SEQ ID NO: 64, (ii) CDR1, CD2, CDR3 domains of the light chain variable region having the sequence set forth in SEQ ID NO: 65, and (iii) a lipocalin mutein with the sequence set forth in any one of SEQ ID NOs: 21-39; and
(b) a pharmaceutical composition comprising a PD-1 axis inhibitor.

37. The kit of parts of claim 36, wherein the CD137/HER2 bispecific agent is at a unit dosage of about 0.05 mg/kg, 0.15 mg/kg, 0.5 mg/kg, 1.0 mg/kg, 2.5 mg/kg, 5.0 mg/kg, or 8.0 mg/kg and the anti-PD-L1 axis inhibitor is at a unit dosage of about 1200 mg.

38. The kit of parts of claim 36 or 37, wherein the pharmaceutical composition comprising an CD137/HER2 bispecific agent and the pharmaceutical composition comprising a PD-1 axis inhibitor are capable of producing additive or synergistic anti-tumor effect.


SEQUENCE LISTING (112 sequences)

SEQ ID NO: 1 - protein, 178 aa, Homo sapiens
QDSTSDLIPAPPLSKVPLQQNFQDNQFQGKWYVVGLAGNAILREDKDPQKMYATIYELKEDKSYNVTSVLFRKKKCDYWIRTFVPGCQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 2 - protein, 178 aa, Artificial
  NGAL98wt
QDSTSDLIPAPPLSKVPLQQNFQDNQFHGKWYVVGLAGNAILREDKDPQKMYATIYELKEDKSYNVTSVLFRKKKCDYWIRTFVPGSQPGEFTLGNIKSYPGLTSYLVRVVSTNYNQHAMVFFKKVSQNREYFKITLYGRTKELTSELKENFIRFSKSLGLPENHIVFPVPIDQCIDG

SEQ ID NO: 3 - protein, 272 aa, Artificial
  human PD-L1 full length
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 4 - protein, 15 aa, Artificial
  Linker
GGGGSGGGGSGGGGS

SEQ ID NO: 5 - protein, 18 aa, Artificial
  Linker
PSTPPTNSSSTPPTPSPS

SEQ ID NO: 6 - protein, 14 aa, Artificial
  Linker
GGSGNSSGSGGSPV

SEQ ID NO: 7 - protein, 20 aa, Artificial
  Linker
ASPAAPAPASPAAPAPSAPA

SEQ ID NO: 8 - protein, 66 aa, Artificial
  Linker
AGSGGSGGSGGSPVPSTPPTPSPSTPPTPSPSGGSGNSSGSGGSPVPSTPPTPSPSTPPTPSPSAS

SEQ ID NO: 9 - protein, 32 aa, Artificial
  Linker
PSTPPTPSPSTPPTPSPSGGSGNSSGSGGSPV

SEQ ID NO: 10 - protein, 74 aa, Artificial
  Linker
AGSGGSGGSGGSPVPSTPPTNSSSTPPTPSPSPVPSTPPTNSSSTPPTPSPSPVPSTPPTNSSSTPPTPSPSAS

SEQ ID NO: 11 - protein, 40 aa, Artificial
  Linker
ASPAAPAPASPAAPAPSAPAASPAAPAPASPAAPAPSAPA

SEQ ID NO: 12 - protein, 10 aa, Artificial
  Linker
VDDIEGRMDE

SEQ ID NO: 13 - protein, 11 aa, Artificial
  Linker
ENLYFQGRMDE

SEQ ID NO: 14 - protein, 10 aa, Artificial
  Linker
GGGGSGGGGS

SEQ ID NO: 15 - protein, 327 aa, Artificial
  hIgG4 HC
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 214 aa, Artificial
  hIgG4 LC
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 228 aa, Artificial
  Fc, IgG4
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 18 - protein, 228 aa, Artificial
  Fc, IgG4
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 228 aa, Artificial
  Fc, IgG4
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLG

SEQ ID NO: 20 - protein, 228 aa, Artificial
  Fc, IgG4
ESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 21 - protein, 178 aa, Artificial
  lipocalin mutein
QDSTSDLIPAPPLSKVPLQQNFQDNQFHGKWYVVGQAGNIKLREDKDPNKMMATIYELKEDKSYNVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 22 - protein, 178 aa, Artificial
  lipocalin mutein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 23 - protein, 178 aa, Artificial
  lipocalin mutein
QDSTSDLIPAPPLSKVPLQQNFQDNQFHGKWYVVGQAGNIRLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 24 - protein, 178 aa, Artificial
  lipocalin mutein
QDSTSDLIPAPPLSKVPLQQNFQDNQFHGKWYVVGQAGNIKLREDKDPNKMMATIYELKEDKSYDVTAVAFDDKKCTYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 25 - protein, 175 aa, Artificial
  lipocalin mutein
QDSTSDLIPAPPLSKVPLQQNFQDNQFHGKWYVVGQAGNIKLREDSKMMATIYELKEDKSYDVTGVSFDDKKCTYAIMTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 26 - protein, 178 aa, Artificial
  lipocalin mutein
QDSTSDLIPAPPLSKVPLQQNFQDNQFHGKWYVVGQAGNIKLREDKDPVKMMATIYELKEDKSYDVTGVTFDDKKCRYDISTFVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 27 - protein, 178 aa, Artificial
  lipocalin mutein
QDSTSDLIPAPPLSKVPLQQNFQDNQFHGKWYVVGQAGNIRLREDKDPHKMMATIYELKEDKSYDVTGVTFDDKKCTYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 28 - protein, 178 aa, Artificial
  lipocalin mutein
QDSTSDLIPAPPLSKVPLQQNFQDNQFHGKWYVVGQAGNIKLREDKDPNKMMATIYELKEDKSYDVTGVTFDDKKCTYAISTLVPGSQPGEFTFGKIKSFPGHTSSLVRVVSTNYNQHAMVFFKFVFQNREEFYITLYGRTKELTSELKENFIRFSKSLGLPENHIVFPVPIDQCIDG

SEQ ID NO: 29 - protein, 178 aa, Artificial
  lipocalin mutein
QDSTSDLIPAPPLSKVPLQQNFQDNQFHGKWYVVGQAGNIRLREDKDPSKMMATIYELKEDKSYDVTAVTFDDKKCNYAISTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 30 - protein, 178 aa, Artificial
  lipocalin mutein
QDSTSDLIPAPPLSKVPLQQNFQDNQFHGKWYVVGMAGNNLLREDKDPHKMSATIYELKEDKSYNVTDVMFLDKKCQYIIWTFVPGSQPGEFTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 31 - protein, 178 aa, Artificial
  lipocalin mutein
QDSTSDLIPAPPLSKVPLQQNFQDNQFQGKWYVVGMAGNNLLREDKDPHKMSATIYELKEDKSYNVTDVMFLDKKCQYIIWTFVPGSQPGELTLGFIRSDLGHTSYLVRVVSTNYNQHAMVFFKSVIQNREWFGITLYGRTKELTSELKENFIRFSKSLGLPENHIVFPVPIDQCIDG

SEQ ID NO: 32 - protein, 178 aa, Artificial
  lipocalin mutein
QDSTSDLIPAPPLSKVPLQQNFQDYQFQGKWYVVGMAGNNLLREDKDPHKMGATIYELKEDKSYNVTDVMLLDKKCQYIIQTFVPGSQPGESTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 33 - protein, 178 aa, Artificial
  lipocalin mutein
QDSTSDLIPAPPLSKVPLQQNFQDNQFQGKWYVVGMAGNNLLREDKDPHKMGATIYELKEDKSYNVTDVMFLDKKCQHIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 34 - protein, 178 aa, Artificial
  lipocalin mutein
QDSTSDLIPAPPLSKVPLQQNFQDDQFQGKWYVVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 35 - protein, 178 aa, Artificial
  lipocalin mutein
QDSTSDLIPAPPLSKVPLQQNFQDNQFQGKWYIVGMAGNNLLREDKDPHKMGATIYELKEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 36 - protein, 178 aa, Artificial
  lipocalin mutein
QDSTSDLIPAPPLSKVPLQRNFQDNQFQGKWYVVGMAGNNLLRVDKDPHKMGATIYELKEDKSYNVTDVMFLDKKCQYIIWTFVPGSQPGELTLGFIKSDPGHTSYLVRVVSTNYNQHAMVYFKSVIQNREWFGITLYGRTKELTSELKENFIRFSKSLGLPENHIVFPVPIDQCIDG

SEQ ID NO: 37 - protein, 178 aa, Artificial
  lipocalin mutein
QDSTSDLIPAPPLSKVPLQQNFQDNQFQGKWYVVGMAGNNLLREDKDPHKMSATIYELKEDKSYNVTDVMFLDKKCQYINWPFVPGSQPGEFTLGFIKSDLGPTSYLVRVVSTNYNQHAMVFFKSVIQNREWFGITLYGRTKELTSELKENFIRFSKSLGLPENHIVFPVPIDQCIDG

SEQ ID NO: 38 - protein, 178 aa, Artificial
  lipocalin mutein
QDSTSDLIPAPPLSKVPLQQNFQDNQFQGKWYVVGMAGNNLLREDKDPHKMGATIYELNEDKSYNVTDVMFLDKKCQYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 39 - protein, 178 aa, Artificial
  lipocalin mutein
QDSTSDLIPAPPLSKVPLQQNFQDNQFQGKWYVVGMAGNNLLRDDKDPHKMSATIYELKEDKSYNVTDVMLLDKKCHYIIWTFVPGSQPGELTLGFIKSDPGHTSYLVRVVSTNYNQHAMVFFKSVIQNREWFGITLYGRTKELTSELKENFIRFSKSLGLPENHIVFPVPIDQCIDG

SEQ ID NO: 40 - protein, 10 aa, Artificial
  Trastuzumab HCDR1
GFNIKDTYIH

SEQ ID NO: 41 - protein, 18 aa, Artificial
  Trastuzumab HCDR2
RIYPTNGYTRYADSVKGR

SEQ ID NO: 42 - protein, 11 aa, Artificial
  Trastuzumab HCDR3
WGGDGFYAMDY

SEQ ID NO: 43 - protein, 12 aa, Artificial
  Trastuzumab LCDR1
RASQDVNTAVAW

SEQ ID NO: 44 - protein, 7 aa, Artificial
  Trastuzumab LCDR2
SASFLYS

SEQ ID NO: 45 - protein, 9 aa, Artificial
  Trastuzumab LCDR3
QQHYTTPPT

SEQ ID NO: 46 - protein, 5 aa, Artificial
  Nivo HCDR1
NSGMH

SEQ ID NO: 47 - protein, 17 aa, Artificial
  Nivo HCDR2
VIWYDGSKRYYADSVKG

SEQ ID NO: 48 - protein, 4 aa, Artificial
  Nivo HCDR3
NDDY

SEQ ID NO: 49 - protein, 11 aa, Artificial
  Nivo LCDR1
RASQSVSSYLA

SEQ ID NO: 50 - protein, 7 aa, Artificial
  Nivo LCDR2
DASNRAT

SEQ ID NO: 51 - protein, 9 aa, Artificial
  Nivo LCDR3
QQSSNWPRT

SEQ ID NO: 52 - protein, 5 aa, Artificial
  Pembro HCDR1
NYYMY

SEQ ID NO: 53 - protein, 16 aa, Artificial
  Pembro HCDR2
GINPSNGGTNFNEKFK

SEQ ID NO: 54 - protein, 11 aa, Artificial
  Pembro HCDR3
RDYRFDMGFDY

SEQ ID NO: 55 - protein, 15 aa, Artificial
  Pembro LCDR1
RASKGVSTSGYSYLH

SEQ ID NO: 56 - protein, 7 aa, Artificial
  Pembro LCDR2
LASYLES

SEQ ID NO: 57 - protein, 9 aa, Artificial
  Pembro LCDR3
QHSRDLPLT

SEQ ID NO: 58 - protein, 10 aa, Artificial
  Atezo HCDR1
GFTFSDSWIH

SEQ ID NO: 59 - protein, 15 aa, Artificial
  Atezo HCDR2
AWISPYGGSTYYADS

SEQ ID NO: 60 - protein, 9 aa, Artificial
  Atez HCDR3
RHWPGGFDY

SEQ ID NO: 61 - protein, 9 aa, Artificial
  Atezo LCDR1
RASQDVSTA

SEQ ID NO: 62 - protein, 7 aa, Artificial
  Atezo LCDR2
SASFLYS

SEQ ID NO: 63 - protein, 9 aa, Artificial
  Atezo LCDR3
QQYLYHPAT

SEQ ID NO: 64 - protein, 120 aa, Artificial [sequence omitted]

SEQ ID NO: 65 - protein, 107 aa, Artificial [sequence omitted]

SEQ ID NO: 66 - protein, 113 aa, Artificial [sequence omitted]

SEQ ID NO: 67 - protein, 107 aa, Artificial [sequence omitted]

SEQ ID NO: 68 - protein, 120 aa, Artificial [sequence omitted]

SEQ ID NO: 69 - protein, 111 aa, Artificial [sequence omitted]

SEQ ID NO: 70 - protein, 118 aa, Artificial [sequence omitted]

SEQ ID NO: 71 - protein, 107 aa, Artificial [sequence omitted]

SEQ ID NO: 72 - protein, 439 aa, Artificial
  Nivolumab HC
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 73 - protein, 214 aa, Artificial; identical to SEQ ID NO: 16
  Nivolumab LC

SEQ ID NO: 74 - protein, 446 aa, Artificial
  Pembroluzumab HC
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 75 - protein, 218 aa, Artificial
  Pembroluzumab LC
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 76 - protein, 448 aa, Artificial
  Atezolizumab HC
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 77 - protein, 214 aa, Artificial
  Atezolizumab LC
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 78 - protein, 450 aa, Artificial
  Trastuzumab-IgG1 HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 447 aa, Artificial
  Trastuzumab-IgG4 HC
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 214 aa, Artificial
  Trastuzumab LC
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 640 aa, Artificial
  fusion protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 82 - protein, 407 aa, Artificial
  fusion protein
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSQDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDG

SEQ ID NO: 83 - protein, 640 aa, Artificial
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 407 aa, Artificial
  fusion protein
QDSTSDLIPAPPLSKVPLQQNFQDNQFHGKWYVVGQAGNIRLREDKDPIKMMATIYELKEDKSYDVTMVKFDDKKCMYDIWTFVPGSQPGEFTLGKIKSFPGHTSSLVRVVSTNYNQHAMVFFKFVFQNREEFYITLYGRTKELTSELKENFIRFSKSLGLPENHIVFPVPIDQCIDGGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 85 - DNA, 534 bp, Homo sapiens
caggatagcaccagcgatctgattccggcgccgccgctgagcaaagtgccgctgcagcagaactttcaggataaccagtttcagggcaaatggtatgtggtgggcctggcgggcaacgcgattctgcgcgaagataaagatccgcagaaaatgtatgcgaccatttatgaactgaaagaagataaaagctataacgtgaccagcgtgctgtttcgcaaaaaaaaatgcgattattggattcgcacctttgtgccgggctgccagccgggcgaatttaccctgggcaacattaaaagctatccgggcctgaccagctatctggtgcgcgtggtgagcaccaactataaccagcatgcgatggtgttttttaaaaaagtgagccagaaccgcgaatattttaaaattaccctgtatggccgcaccaaagaactgaccagcgaactgaaagaaaactttattcgctttagcaaaagcctgggcctgccggaaaaccatattgtgtttccggtgccgattgatcagtgcattgatggc

SEQ ID NO: 86 - DNA, 534 bp, Artificial
  NGAL98wt
caggatagcaccagcgatctgattccggcgccgccgctgagcaaagtgccgctgcagcagaactttcaggataaccagtttcatggcaaatggtatgtggtgggcctggcgggcaacgcgattctgcgcgaagataaagatccgcagaaaatgtatgcgaccatttatgaactgaaagaagataaaagctataacgtgaccagcgtgctgtttcgcaaaaaaaaatgcgattattggattcgcacctttgtgccgggcagccagccgggcgaatttaccctgggcaacattaaaagctatccgggcctgaccagctatctggtgcgcgtggtgagcaccaactataaccagcatgcgatggtgttttttaaaaaagtgagccagaaccgcgaatattttaaaattaccctgtatggccgcaccaaagaactgaccagcgaactgaaagaaaactttattcgctttagcaaaagcctgggcctgccggaaaaccatattgtgtttccggtgccgattgatcagtgcattgatggc

SEQ ID NO: 87 - DNA, 534 bp, Artificial
  lipocalin mutein
caggactccacctcagacctgatcccagccccacctctgagcaaggtccctctgcagcagaacttccaggacaaccaattccatgggaagtggtatgtggtaggtcaggcagggaatatcaaactcagagaagacaaagacccgaacaagatgatggccaccatctatgagctgaaagaagacaagagctacaatgtcaccggtgtcacttttgacgacaagaagtgtacttacgctatctctacttttgttccaggttcccagccaggcgagttcacgctgggcaaaattaagagtttccctggacatacgagttctctcgtccgagtggtgagcaccaactacaaccagcatgctatggtgttcttcaagttcgttttccaaaacagggaggaattctacatcaccctctacgggagaaccaaggagctgacttcggaactaaaggagaacttcatccgcttctccaaatctctgggcctccctgaaaaccacatcgtcttccctgtcccaatcgaccagtgtatcgacggc

SEQ ID NO: 88 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaggctgcgtgaggataaggatccgattaaaatgatggcgaccatttacgagttgaaagaagataaatcatatgacgtcaccatggtgaagtttgatgataagaaatgcatgtacgatatttggacctttgtgccggggagccagccgggcgagtttactttaggcaagattaaaagttttccgggccatacatcatcgttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 89 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaggctgcgtgaggataaggatccgaataaaatgatggcgaccatttacgagttgaaagaagataaatcatatgacgtcaccgcggtggcgtttgatgataagaaatgcacgtacgatatttggacctttgtgccggggagccagccgggcgagtttactttaggcaagattaaaagttttccgggccatacatcatcgttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 90 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaagctgcgtgaggataaggatccgaataaaatgatggcgaccatttacgagttgaaagaagataaatcatatgacgtcaccgcggtggcgtttgatgataagaaatgcacgtacgatatttggacctttgtgccggggagccagccgggcgagtttactttaggcaagattaaaagttttccgggccatacatcatctttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 91 - DNA, 525 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaagctgcgtgaggatagtaaaatgatggcgaccatttacgagttgaaagaagataaatcatatgacgtcaccggtgtgagttttgatgataagaaatgcacgtacgctattatgacctttgtgccggggagccagccgggcgagtttactttaggcaagattaaaagttttccgggccatacatcatcgttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 92 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaagctgcgtgaggataaggatccggttaaaatgatggcgaccatttacgagttgaaagaagataaatcatatgacgtcaccggggtgacgtttgatgataagaaatgcaggtacgatatttcgacctttgtgccggggagccagccgggcgagtttacttttggcaagattaaaagttttccgggccatacatcatcgttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 93 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaggctgcgtgaggataaggatccgcataaaatgatggcgaccatttacgagttgaaagaagataaatcatatgacgtcaccggggtgacttttgatgataagaaatgcacgtacgctatttcgacctttgtgccggggagccagccgggcgagtttactttaggcaagattaaaagttttccgggccatacatcatctttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 94 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaagctgcgtgaggataaggatccgaataaaatgatggcgaccatttacgagttgaaagaagataaatcatatgacgtcaccggggtgacttttgatgataagaaatgcacgtacgctatttctacccttgtgccggggagccagccgggcgagtttacttttggcaagattaaaagttttccgggccatacatcatcgttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 95 - DNA, 534 bp, Artificial
  lipocalin mutein
caggactccacctcagacctgatcccagccccacctctgagcaaggtccctctgcagcagaacttccaggacaaccaattccatgggaaatggtacgttgtcgggcaggccggaaatattaggctgcgtgaggataaggatccgtctaaaatgatggcgaccatttacgagttgaaagaagataaatcatatgacgtcaccgctgtgacgtttgatgataagaaatgcaattacgctatttctacctttgtgccggggagccagccgggcgagtttactttaggcaagattaaaagttttccgggccatacatcatcgttggtccgcgtcgtgagcaccaactacaaccagcatgccatggtgttcttcaagtttgtgtttcagaaccgcgaggagttttatatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 96 - DNA, 534 bp, Artificial
  lipocalin mutein
caggactccacctcagacctgatcccagccccacctctgagcaaggtccctctgcagcagaacttccaggacaaccaattccatgggaaatggtatgtcgtgggcatggccggaaataatctgctgcgtgaggataaggatccgcacaaaatgagcgcgaccatttacgagttgaaagaagataaatcatataacgtcaccgacgtgatgtttctggacaagaaatgccaatacatcatttggacctttgtgccggggagccagccgggcgagtttactttaggcttcattaaaagtgacccgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggc

SEQ ID NO: 97 - DNA, 535 bp, Artificial
  lipocalin mutein
caggactccacctcagacctgatcccagccccacctctgagcaaggtccctctgcagcagaacttccaggacaaccaattccaagggaaatggtatgtcgtgggcatggccggaaataatctgctgcgtgaggataaggatccgcacaaaatgagcgcgaccatttacgagttgaaagaagataaatcatataacgtcaccgacgtgatgtttctggacaagaaatgccaatacatcatttggacctttgtgccggggagccagccgggcgagcttactttaggcttcattagaagtgacctgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 98 - DNA, 535 bp, Artificial
  lipocalin mutein
caggactccacctcagacctgatccctgccccacctctgagcaaggtccctctgcagcagaacttccaggactaccaattccaagggaaatggtatgtcgtgggcatggccggaaataatctgctgcgtgaggataaggatccgcacaaaatgggcgcgaccatttacgagttgaaagaagataaatcatataacgtcaccgacgtgatgcttctggacaagaaatgccaatacatcattcagacctttgtgccggggagccagccgggcgagtctactttaggcttcattaaaagtgacccgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 99 - DNA, 535 bp, Artificial
  lipocalin mutein
caggactccacctcagacctgatcccagccccacctctgagcaaggtccctctgcagcagaacttccaggacaaccaattccaagggaaatggtatgtcgtgggcatggccggaaataatctgctgcgtgaggataaggatccgcacaaaatgggcgcgaccatttacgagttgaaagaagataaatcatataacgtcaccgacgtgatgtttctggacaagaaatgccaacacatcatatggacctttgtgccggggagccagccgggcgagttaactttaggcttcattaaaagtgacccgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 100 - DNA, 535 bp, Artificial
  lipocalin mutein
caggactccacctcagacctgatcccagccccacctctgagcaaggtccctctgcagcagaacttccaggacgaccaattccaagggaaatggtatgtcgtgggcatggccggaaataatctgttgcgtgaggataaggatccgcacaaaatgggcgcgaccatttacgagttgaaagaagataaatcatataacgtcaccgacgtgatgtttctggacaagaaatgccaatacatcatttggacctttgtgccggggagccagccgggcgagttgactttaggcttcattaaaagtgacccgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 101 - DNA, 535 bp, Artificial
  lipocalin mutein
caggactccacctcagacctgatcccagccccacctctgagcaaggtccctctgcagcagaacttccaggacaaccaattccaagggaaatggtatatcgtgggcatggccggaaataatctgctgcgtgaggataaggatccgcacaaaatgggcgcgaccatttacgagttgaaagaagataaatcatataacgtcaccgacgtgatgtttctggacaagaaatgccaatacatcatttggacctttgtgccggggagccagccgggcgagcttactttaggcttcattaaaagtgacccgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 102 - DNA, 535 bp, Artificial
  lipocalin mutein
caggactccacctcagacctgatcccagccccacctctgagcaaggtccctctgcagcggaacttccaggacaaccaattccaagggaagtggtatgtcgtgggcatggccggaaataatctgctgcgtgtggataaggatccgcacaaaatgggcgcgaccatttacgagttgaaagaagataaatcatataacgtcaccgacgtgatgtttctggacaagaaatgccaatacatcatttggacctttgtgccggggagccagccgggcgagttaactttaggcttcattaaaagtgacccgggccacacatcatacttggtccgcgtcgtgagcaccaactacaaccagcatgccatggtgtacttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 103 - DNA, 535 bp, Artificial
  lipocalin mutein
caggactccacctcagatctgatcccagccccacctctgagcaaggtccctctgcagcagaacttccaggacaaccaattccaagggaaatggtatgtcgtgggcatggccggaaataatctgctgcgtgaggataaggatccgcacaaaatgagcgcgaccatttacgagttgaaagaagataaatcatataacgtcaccgacgtgatgtttctggacaagaaatgccaatacatcaattggccctttgtgccggggagccagccgggcgagtttactttaggcttcattaaaagtgacctgggccccacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 104 - DNA, 535 bp, Artificial
  lipocalin mutein
caggactccacctcagacctgatcccagccccacctctgagcaaggtccctctgcagcagaacttccaggacaaccaattccaagggaaatggtatgtcgtgggcatggccggaaataatctgctgcgtgaggataaggatccgcacaaaatgggtgcgaccatttacgagttgaatgaagataaatcatataacgtcaccgacgtgatgtttctggacaagaaatgccaatacatcatttggacctttgtgccggggagccagccgggcgagcttactttaggcttcattaaaagtgacccgggccacacatcatacttggtccgcgtcgtgagcaccaactacaaccagcatgccatggtgttcttcaagagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 105 - DNA, 535 bp, Artificial
  lipocalin mutein
caggactccacctcagacctgatcccagccccacctctgagcaaggtccctctgcagcagaacttccaggacaaccaattccaagggaaatggtatgtcgtgggcatggccggaaataatctgctgcgtgatgataaggatccgcacaaaatgagcgctaccatttacgagttgaaagaagataaatcatataacgtcaccgacgtgatgttactggacaagaaatgccattacatcatttggacctttgtgccggggagccagccgggcgagcttactttaggcttcattaaaagtgacccgggccacacatcatacttggtccgcgtcgtgagcaccaactacaaccagcatgccatggtgttcttcaaaagcgtgatccagaaccgcgagtggtttggaatcacactgtacgggcgcacgaaagaactgacaagcgagctgaaggaaaattttatccgcttttccaaatctctgggcctccctgaaaaccacatcgtcttccctgtcccaatcgaccagtgtatcgacggca

SEQ ID NO: 106 - DNA, 1350 bp, Artificial
  Trastuzumab-IgG1 HC
gaagtccagctggtcgaatctggtggtggcctggtccagcctggtggatcactgagactgtcctgtgctgcttctggtttcaacatcaaggacacctacatccattgggtcagacaggcacctggcaagggactggaatgggtcgcccgaatctaccctacaaacggctacactcgctacgccgactccgtcaagggacgctttaccatctccgccgacacctctaaaaacaccgcctacctgcagatgaatagtctgagggccgaggatactgctgtgtactactgctcacgatggggaggcgacggcttttacgctatggattactggggacagggaactctggtcactgtgtctagcgctagcacaaagggccctagtgtgtttcctctggctccctcttccaaatccacttctggtggcactgctgctctgggatgcctggtgaaggattactttcctgaacctgtgactgtctcatggaactctggtgctctgacttctggtgtccacactttccctgctgtgctgcagtctagtggactgtactctctgtcatctgtggtcactgtgccctcttcatctctgggaacccagacctacatttgtaatgtgaaccacaaaccatccaacactaaagtggacaaaaaagtggaacccaaatcctgtgacaaaacccacacctgcccaccttgtcctgcccctgaactgctgggaggaccttctgtgtttctgttcccaccaaaaccaaaagataccctgatgatctctagaacccctgaggtgacatgtgtggtggtggatgtgtctcatgaggaccctgaggtcaaattcaactggtacgtggatggagtggaagtccacaatgccaaaaccaagcctagagaggaacagtacaattcaacctacagagtggtcagtgtgctgactgtgctgcatcaggattggctgaatggcaaggaatacaagtgtaaagtctcaaacaaggccctgcctgctccaattgagaaaacaatctcaaaggccaagggacagcctagggaaccccaggtctacaccctgccaccttcaagagaggaaatgaccaaaaaccaggtgtccctgacatgcctggtcaaaggcttctacccttctgacattgctgtggagtgggagtcaaatggacagcctgagaacaactacaaaacaaccccccctgtgctggattctgatggctctttctttctgtactccaaactgactgtggacaagtctagatggcagcaggggaatgtcttttcttgctctgtcatgcatgaggctctgcataaccactacactcagaaatccctgtctctgtctcctggcaaa

SEQ ID NO: 107 - DNA, 1341 bp, Artificial
  Trastuzumab-IgG4 HC
gaagtccagctggtcgaatctggtggtggcctggtccagcctggtggatcactgagactgtcctgtgctgcttctggtttcaacatcaaggacacctacatccattgggtcagacaggcacctggcaagggactggaatgggtcgcccgaatctaccctacaaacggctacactcgctacgccgactccgtcaagggacgctttaccatctccgccgacacctctaaaaacaccgcctacctgcaaatgaatagtctgagggccgaggatactgctgtgtactactgctcacgatggggaggcgacggcttttacgctatggattactggggacagggaactctggtcactgtctcgagcgctagcaccaagggcccctccgtgttccccctggccccttgctcccggtccacctccgagtctaccgccgctctgggctgcctggtgaaagactacttccccgagcctgtgaccgtgagctggaactctggcgccctgacctccggcgtgcacaccttccctgccgtgctgcaatcctccggcctgtactccctgtcctccgtggtgacagtgccctcctccagcctgggcaccaagacctacacctgtaacgtggaccacaagccctccaacaccaaggtggacaagcgggtggaatctaaatacggccctccctgccccccctgccctgcccctgaagcggcgggcggaccttccgtgtttctgttccccccaaagcccaaggacaccctgatgatctcccggacccccgaagtgacctgcgtggtggtggacgtgtcccaggaagatccagaggtgcagttcaactggtatgttgacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtgaaaggcttctacccctccgacattgccgtggaatgggagtccaacggccagcccgagaacaactacaagaccaccccccctgtgctggactccgacggctccttcttcctgtactctcggctgacagtggataagtcccggtggcaggaaggcaatgtgttctcctgcagcgtgatgcacgaggccctgcacaaccactatacccagaagtccctgtccctgagcctgggcaag

SEQ ID NO: 108 - DNA, 642 bp, Artificial
  Trastuzumab LC
gacatccagatgacacagtctccctcttccctgtccgcttctgtgggcgatcgagtgacaatcacctgtagggctagtcaggatgtgaatactgctgttgcttggtaccagcagaaaccaggaaaagcccctaaactgctgatctactctgcctcattcctgtactctggggtgccttctcgattcagtggttctagatctggcaccgatttcacactgaccatttcttcactgcaacctgaggattttgccacctactactgtcagcagcactacacaacacctcccacatttggccagggcacaaaagtggagatcaaacggaccgtggcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgc

SEQ ID NO: 109 - DNA, 1920 bp, Artificial
  fusion protein
gaagtccagctggtcgaatctggtggtggcctggtccagcctggtggatcactgagactgtcctgtgctgcttctggtttcaacatcaaggacacctacatccattgggtcagacaggcacctggcaagggactggaatgggtcgcccgaatctaccctacaaacggctacactcgctacgccgactccgtcaagggacgctttaccatctccgccgacacctctaaaaacaccgcctacctgcaaatgaatagtctgagggccgaggatactgctgtgtactactgctcacgatggggaggcgacggcttttacgctatggattactggggacagggaactctggtcactgtctcgagcgctagcaccaagggcccctccgtgttccccctggccccttgctcccggtccacctccgagtctaccgccgctctgggctgcctggtgaaagactacttccccgagcctgtgaccgtgagctggaactctggcgccctgacctccggcgtgcacaccttccctgccgtgctgcaatcctccggcctgtactccctgtcctccgtggtgacagtgccctcctccagcctgggcaccaagacctacacctgtaacgtggaccacaagccctccaacaccaaggtggacaagcgggtggaatctaaatacggccctccctgccccccctgccctgcccctgaagcggcgggcggaccttccgtgtttctgttccccccaaagcccaaggacaccctgatgatctcccggacccccgaagtgacctgcgtggtggtggacgtgtcccaggaagatccagaggtgcagttcaactggtatgttgacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtgaaaggcttctacccctccgacattgccgtggaatgggagtccaacggccagcccgagaacaactacaagaccaccccccctgtgctggactccgacggctccttcttcctgtactctcggctgacagtggataagtcccggtggcaggaaggcaatgtgttctcctgcagcgtgatgcacgaggccctgcacaaccactatacccagaagtccctgtccctgagcctgggcaagggcggtggaggatccgggggtgggggaagcggcggaggaggtagccaggactctactagtgatctgatcccggcaccgccactgtcaaaagtccctctgcaacaaaactttcaagacaatcagtttcacggcaaatggtatgtggtcggccaggccggaaacattaggctgcgggaggacaaggaccccatcaaaatgatggctaccatctacgagctgaaggaagacaaatcttatgatgtgacaatggtcaagttcgacgataagaaatgcatgtacgacatctggaccttcgtgcccggctcccagccgggagagttcaccctgggcaagatcaagtccttccccggccacacttccagcctggtccgcgtggtctcgaccaactataatcagcatgctatggtgttcttcaagttcgtctttcagaatagagaggagttctacatcacactgtatggacgcaccaaggagctgacaagcgagctgaaagaaaacttcatcaggttttcaaagtccctggggctgcccgaaaatcatatcgtgttcccagtccccatcgaccagtgtattgatggt

SEQ ID NO: 110 - DNA, 1221 bp, Artificial
  fusion protein
gacatccagatgacacagtctccctcttccctgtccgcttctgtgggcgatcgagtgacaatcacctgtagggctagtcaggatgtgaatactgctgttgcttggtaccagcagaaaccaggaaaagcccctaaactgctgatctactctgcctcattcctgtactctggggtgccttctcgattcagtggttctagatctggcaccgatttcacactgaccatttcttcactgcaacctgaggattttgccacctactactgtcagcagcactacacaacacctcccacatttggccagggcacaaaagtggagatcaaacggaccgtggcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgcggcggcggaggatccgggggtgggggaagcggcggaggaggtagccaggactctactagtgatctgatcccggcaccgccactgtcaaaagtccctctgcaacaaaactttcaagacaatcagtttcacggcaaatggtatgtggtcggccaggccggaaacattaggctgcgggaggacaaggaccccatcaaaatgatggctaccatctacgagctgaaggaagacaaatcttatgatgtgacaatggtcaagttcgacgataagaaatgcatgtacgacatctggaccttcgtgcccggctcccagccgggagagttcaccctgggcaagatcaagtccttccccggccacacttccagcctggtccgcgtggtctcgaccaactataatcagcatgctatggtgttcttcaagttcgtctttcagaatagagaggagttctacatcacactgtatggacgcaccaaggagctgacaagcgagctgaaagaaaacttcatcaggttttcaaagtccctggggctgcccgaaaatcatatcgtgttcccagtccccatcgaccagtgtattgatggt

SEQ ID NO: 111 - DNA, 1920 bp, Artificial
  fusion protein
caggactctactagtgatctgatcccggcaccgccactgtcaaaagtccctctgcaacaaaactttcaagacaatcagtttcacggcaaatggtatgtggtcggccaggccggaaacattaggctgcgggaggacaaggaccccatcaaaatgatggctaccatctacgagctgaaggaagacaaatcttatgatgtgacaatggtcaagttcgacgataagaaatgcatgtacgacatctggaccttcgtgcccggctcccagccgggagagttcaccctgggcaagatcaagtccttccccggccacacttccagcctggtccgcgtggtctcgaccaactataatcagcatgctatggtgttcttcaagttcgtctttcagaatagagaggagttctacatcacactgtatggacgcaccaaggagctgacaagcgagctgaaagaaaacttcatcaggttttcaaagtccctggggctgcccgaaaatcatatcgtgttcccagtccccatcgaccagtgtattgatggaggaggcggaggatccggcggaggaggaagtggcggaggaggaagtgaagtccagctggtcgaatctggtggtggcctggtccagcctggtggatcactgagactgtcctgtgctgcttctggtttcaacatcaaggacacctacatccattgggtcagacaggcacctggcaagggactggaatgggtcgcccgaatctaccctacaaacggctacactcgctacgccgactccgtcaagggacgctttaccatctccgccgacacctctaaaaacaccgcctacctgcagatgaatagtctgagggccgaggatactgctgtgtactactgctcacgatggggaggcgacggcttttacgctatggattactggggacagggaactctggtcactgtgtctagcgctagcaccaagggcccctccgtgttccccctggccccttgctcccggtccacctccgagtctaccgccgctctgggctgcctggtgaaagactacttccccgagcctgtgaccgtgagctggaactctggcgccctgacctccggcgtgcacaccttccctgccgtgctgcaatcctccggcctgtactccctgtcctccgtggtgacagtgccctcctccagcctgggcaccaagacctacacctgtaacgtggaccacaagccctccaacaccaaggtggacaagcgggtggaatctaaatacggccctccctgccccccctgccctgcccctgaagcggcgggcggaccttccgtgtttctgttccccccaaagcccaaggacaccctgatgatctcccggacccccgaagtgacctgcgtggtggtggacgtgtcccaggaagatccagaggtgcagttcaactggtatgttgacggcgtggaagtgcacaacgccaagaccaagcccagagaggaacagttcaactccacctaccgggtggtgtccgtgctgaccgtgctgcaccaggactggctgaacggcaaagagtacaagtgcaaggtgtccaacaagggcctgccctccagcatcgaaaagaccatctccaaggccaagggccagccccgcgagccccaggtgtacaccctgccccctagccaggaagagatgaccaagaaccaggtgtccctgacctgtctggtgaaaggcttctacccctccgacattgccgtggaatgggagtccaacggccagcccgagaacaactacaagaccaccccccctgtgctggactccgacggctccttcttcctgtactctcggctgacagtggataagtcccggtggcaggaaggcaatgtgttctcctgcagcgtgatgcacgaggccctgcacaaccactatacccagaagtccctgtccctgagcctgggcaag

SEQ ID NO: 112 - DNA, 1221 bp, Artificial
  fusion protein
caggactctactagtgatctgatcccggcaccgccactgtcaaaagtccctctgcaacaaaactttcaagacaatcagtttcacggcaaatggtatgtggtcggccaggccggaaacattaggctgcgggaggacaaggaccccatcaaaatgatggctaccatctacgagctgaaggaagacaaatcttatgatgtgacaatggtcaagttcgacgataagaaatgcatgtacgacatctggaccttcgtgcccggctcccagccgggagagttcaccctgggcaagatcaagtccttccccggccacacttccagcctggtccgcgtggtctcgaccaactataatcagcatgctatggtgttcttcaagttcgtctttcagaatagagaggagttctacatcacactgtatggacgcaccaaggagctgacaagcgagctgaaagaaaacttcatcaggttttcaaagtccctggggctgcccgaaaatcatatcgtgttcccagtccccatcgaccagtgtattgatggaggaggcggaggatccggcggaggaggaagtggcggaggaggaagtgacatccagatgacacagtctccctcttccctgtccgcttctgtgggcgatcgagtgacaatcacctgtagggctagtcaggatgtgaatactgctgttgcttggtaccagcagaaaccaggaaaagcccctaaactgctgatctactctgcctcattcctgtactctggggtgccttctcgattcagtggttctagatctggcaccgatttcacactgaccatttcttcactgcaacctgaggattttgccacctactactgtcagcagcactacacaacacctcccacatttggccagggcacaaaagtggagatcaaacggaccgtggcggcgccttctgtgttcattttccccccatctgatgaacagctgaaatctggcactgcttctgtggtctgtctgctgaacaacttctaccctagagaggccaaagtccagtggaaagtggacaatgctctgcagagtgggaattcccaggaatctgtcactgagcaggactctaaggatagcacatactccctgtcctctactctgacactgagcaaggctgattacgagaaacacaaagtgtacgcctgtgaagtcacacatcaggggctgtctagtcctgtgaccaaatccttcaataggggagagtgc